(19) United States
(12) Patent Application Publication
(10) Pub. No.: US 2015/0086982 A1
(43) Pub. Date: Mar. 26, 2015

(54) COMPOSITIONS AND METHODS FOR DETECTING AND DISCRIMINATING BETWEEN YEAST OR MOLD

(71) Applicants: Michael McClellan Becker, San Diego, CA (US); Bettina Groschel, San Diego, CA (US); Anja Bubeck-Barrett, Encinitas, CA (US)

(72) Inventors: Michael McClellan Becker, San Diego, CA (US); Bettina Groschel, San Diego, CA (US); Anja Bubeck-Barrett, Encinitas, CA (US)

(73) Assignee: ROKA BIOSCIENCE, INC., Warren, NJ (US)

(21) Appl. No.: 14/239,322

(22) PCT Filed: Aug. 17, 2012
(86) PCT No.: PCT/US12/51423

§ 371 Date: Nov. 7, 2014

Related U.S. Application Data
(60) Provisional application No. 61/525,317, filed on Aug. 19, 2011.

(51) Int. Cl. C12Q 1/68

(52) U.S. Cl. CPC C12Q 1/6895; C12Q 2600/158
USPC 435/6.11

(57) ABSTRACT

The invention features compositions, methods, and kits for detecting and distinguishing between yeast or mold in a sample suspected of being contaminated with yeast or mold.

### CROSS-REFERENCE TO RELATED APPLICATIONS

This application claims benefit of U.S. Provisional Application No. 61/525,317, filed Aug. 19, 2011, the contents of which are hereby incorporated by reference in their entirety.

### FIELD OF THE INVENTION

The invention features compositions, methods, and kits for detecting and discriminating between the presence of mold and yeast in a sample (e.g., from a food, water, or environmental product or process, an industrial product or process, a pharmaceutical product or process, or a biological tissue, such as blood). The invention features probes and primers that specifically hybridize to mold or yeast nucleic acid molecules (e.g., endogenous chitin synthase genes or ribonucleic acid (RNA) molecules). The primers can be used in conjunction with a transcription-mediated amplification (TMA) assay or a polymerase chain reaction (PCR) assay. The amplified DNA or RNA products generated by the primers can be assayed using mold-specific or yeast or mold-specific probes that can be used to confirm the presence of mold only, mold and yeast, or yeast only in the sample. Thus, the primers and probes of the present invention allow for early detection of, and discrimination between, yeast or mold, in samples suspected of containing these contaminants, relative to the current standard method of culturing from such samples.

### BACKGROUND OF THE INVENTION

Diagnosing mold and/or yeast infection or infiltration/contamination continues to be a major challenge in clinical, manufacturing, and environmental settings. In addition to the time required to grow the mold or yeast from samples, such as a blood culture or manufacturing production samples or reagents, additional time is needed to identify and speciate the organism. All of this testing can become a very lengthy process, which leaves the infection or contamination unchecked and may lead to an increased fungal load or presence.

In addition, most regulatory agencies require, and customers demand, specific testing for contaminating microorganisms that are common to specific food types and agricultural products, prevalent in the utility (e.g., water and wastewater treatment) and pharmaceutical industries, and that are capable of in vivo multiplication. Rapid and accurate methods for detection of foodborne and waterborne contaminating microorganisms are essential, particularly in the context of food manufacturing processes, pharmaceutical industry, drinking water, and other regulated industrial processes. The same applies to the general detection of human, animal, and plant pathogens. Many manufacturers and utilities have, consequently, had to build in-house labs to expedite the testing, or lose valuable time waiting for test results when samples are shipped out to outside labs. Furthermore, using art-recognized and current standard methods, the cost of enrichment media used to expand the numbers of one or more particular microorganisms to detectable levels can be substantial.

Thus, there remains a need in the art for methods and kits for detecting the presence of, and discriminating between, microorganisms, such as yeast or mold in a sample in a timely and efficient manner and, when necessary, in remote test locations.

### SUMMARY OF THE INVENTION

The present invention relates to compositions, reaction mixtures, kits and methods for amplifying and detecting nucleic acids from various species of fungi, including compositions and methods of the invention can be used to distinguish between yeast or mold in a sample.

Accordingly, the invention features a method for detecting and distinguishing between yeast or mold in a sample suspected of being contaminated with the yeast or mold including: a) contacting nucleic acid molecules from the sample with a mold-specific probe or a yeast or mold-specific probe under hybridization conditions, where the mold-specific probe is capable of specifically binding to a mold-specific region including at least 17 (e.g., at least 18, 19, 20, 21, 22, 23, 24, or 25) contiguous base pairs within an endogenous chitin synthase gene of the mold but not to a yeast or mold-specific region of the endogenous chitin synthase gene, where the mold-specific region shares greater than 72% (e.g., greater than about 75%, greater than about 80%, greater than about 85%, greater than about 90%, or greater than about 95%) sequence identity between two or more (e.g., at least 3, 5, 10, 15, or 20 or more, e.g., up to 50 or more) different species of the mold, and where the yeast or mold-specific probe is capable of specifically binding to a yeast or mold-specific region including at least 17 (e.g., at least 18, 19, 20, 21, 22, 23, 24, or 25) contiguous base pairs within an endogenous 1,3-β-glucan synthase gene of the yeast or mold, where the yeast or mold-specific region shares greater than 70% (e.g., greater than about 75%, greater than about 80%, greater than about 85%, greater than about 90%, or greater than about 95%) sequence identity between two or more (e.g., at least 3, 5, 10, 15, or 20 or more, e.g., up to 50 or more) different species of the yeast or mold; and b) detecting hybridization between the mold-specific or the yeast or mold-specific probe and the nucleic acid molecules, where hybridization of the mold-specific probe with the nucleic acid molecule indicates the presence of the mold in the sample; or hybridization of the yeast or mold-specific probe with the nucleic acid molecule indicates the presence of the yeast or mold in the sample. In some embodiments, the endogenous chitin synthase gene is of class I, II, III, IV, V, VI, or VII (e.g., class III, such as any sequence having at least 70% (e.g., at least 80%, 85%, 90%, 95%, 98%, 99%, or even 100%) sequence identity to a sequence of any one of SEQ ID NOs:1-64). In some embodiments, endogenous 1,3-β-glucan synthase gene is any sequence having at least 70% (e.g., at least 80%, 85%, 90%, 95%, 98%, 99%, or even 100%) sequence identity to a sequence of any one of

In some embodiments, the endogenous chitin synthase gene specifically bound by the mold-specific probe is a chitin synthase class III gene. In some embodiments, the mold-specific region includes at least about 17 (e.g., at least 18, 19, 20, 21, 22, 23, 24, or 25) contiguous base pairs within the region of nucleotides 2859 to 2968 of the chitin synthase class III gene, or a ribonucleic acid (RNA) product thereof.

In some embodiments, the endogenous 1,3-β-glucan synthase gene specifically bound by the yeast or mold-specific probe is a 1,3-β-glucan synthase gene. In some embodiments, the yeast or mold-specific region includes at least about 17 (e.g., at least 18, 19, 20, 21, 22, 23, 24, or 25) contiguous base pairs within the region of nucleotides 5360 to 5515 of the 1,3-β-glucan synthase 1 gene, or a ribonucleic acid (RNA) product thereof.

In some embodiments, the method includes incubating the mold-specific and yeast or mold-specific probes with the nucleic acid molecules under hybridization conditions (e.g., stringent conditions, such as any described herein), where hybridization of the mold-specific probe and the yeast or mold-specific probe with the nucleic acid molecules indicates the presence of the mold only or the yeast and the mold in the sample; and hybridization of the mold-specific probe, but not the yeast or mold-specific probe, with the nucleic acid molecule indicates the presence of the mold but not the yeast in the sample; and hybridization of the yeast or mold-specific probe, with the nucleic acid molecule indicates the presence of the yeast or mold. In some embodiments, a difference in hybridization signal intensity (e.g., a difference in intensity of at least about 2-fold, about 5-fold, about 10-fold, about 50-fold, about 100-fold, about 500-fold, about 1,000-fold, about 5,000-fold, about 10,000-fold, or more) between the mold-specific probe relative to the yeast or mold-specific probe indicates the presence of the yeast in the sample.

In any of the embodiments described herein, prior to contacting step a), the method includes amplifying the nucleic acid molecules from the sample. In some embodiments, amplifying includes: (a) contacting the sample including the nucleic acid molecules with a first amplification primer having a target sequence specific for a 5′ region of the endogenous chitin synthase gene or RNA of the mold and/or a second amplification primer having a target sequence specific for a 5′ region of the 1,3-β-glucan synthase gene or RNA; and (b) performing a nucleic acid amplification reaction to produce a first and/or second amplification product, respectively. In particular embodiments, the first and/or second amplification product includes a region of at least about 15 (e.g., at least about 20, about 30, about 40, about 50, about 60, or more) contiguous base pairs that are conserved between different fungal microorganisms. In some embodiments, the region is conserved between different yeast. In other embodiments, the region is conserved between different molds. In some embodiments, the region includes about 20 to about 100 (e.g., at least about 30, about 40, about 50, about 60, about 70, about 80, about 90, or about 100) base pairs.

In some embodiments, the mold-specific probe includes a sequence having at least 70% (e.g., at least about 80%, about 85%, about 90%, about 95%, about 99%, or more, even 100%) sequence identity to a complementary sequence including at least 17 (e.g., at least 18, 19, 20, 21, 22, 23, 24, or 25) contiguous nucleotides within the sequence of SEQ ID NO:153.

In some embodiments, the mold-specific probe includes a sequence having at least 70% (e.g., at least about 80%, about 85%, about 90%, about 95%, about 99%, or more, even 100%) sequence identity to a complementary sequence including 17 to 100 (e.g., at least about 20, about 25, about 30, about 35, about 40, about 45, about 50, about 55, about 60, about 65, about 70, about 75, about 80, about 85, about 90, or about 95) contiguous nucleotides within nucleotides 2850 to 2970 of the sequence of any one of SEQ ID NOs:1-42.

In some embodiments, the yeast or mold-specific probe includes a sequence having at least 70% (e.g., at least about 80%, about 85%, about 90%, about 95%, about 99%, or more, even 100%) sequence identity to a complementary sequence including at least 15 (e.g., at least 15 to 60, or at least 17, 18, 19, 20, 21, 22, 23, 24, 25, 30, 35, 40, 45, 50, 55, or 60) contiguous nucleotides within the sequence of SEQ ID NO:154.

In some embodiments, the yeast or mold-specific probe includes a sequence having at least 70% (e.g., at least about 80%, about 85%, about 90%, about 95%, about 99%, or more, even 100%) sequence identity to a complementary sequence including 17 to 100 (e.g., at least about 20, about 25, about 30, about 35, about 40, about 45, about 50, about 55, about 60, about 65, about 70, about 75, about 80, about 85, about 90, or about 95) contiguous nucleotides within nucleotides 5360 to 5515 of the sequence of any one of SEQ ID NOs:65-125.

In any of the embodiments described herein, the yeast or mold-specific probe binds to the yeast or mold-specific region within the yeast and mold.

In any of the embodiments described herein, the method distinguishes between the presence of the mold and the yeast in the sample.

In any of the embodiments described herein, the mold is selected from phylum Ascomycota (e.g., Ajellomyces spp., Alternaria spp., Aschersonia spp., Aspergillus spp., Arthroderma spp., Ascochyta spp., Bipolaris spp., Blastomyces spp., Botryotinia spp., Chaetomium spp., Cladosporium spp., Coccidioides spp., Curvularia spp., Emericella spp., Emmonsia spp., Epicoccum spp., Exophiala spp., Fusarium spp., Geomyces spp., Geotrichum spp., Gibberella spp., Histoplasma spp., Magnaporthe spp., Metarhizium spp., Monascus spp., Mycospaerella spp., Nectria spp., Neosartorya spp., Neurospora spp., Paecilomyces spp., Paracoccidioides spp., Penicillium spp., Phaeosphaeria spp., Phialemonium spp., Podospora spp., Pyrenophora spp., Sclerotinia spp., Scopulariopsis spp., Sporothrix spp., Stachybotrys spp., Stemphylium spp., Talaromyces spp., Trichophyton spp., Trichothecium spp., Tricoderma spp., Tuber spp., Uncinocarpus spp., or Verticillium spp.), phylum Basidomycota (e.g., Moniliophthora spp., Sporobolomyces spp., Trichosporon spp., Ustilago spp., Cryptococcus spp. or Rhodotorula spp.),), phylum Chytridiomycota, phylum Zygomycota (e.g., Absidia spp., Amylomyces spp., Pilaira spp., Rhizomucor spp., Rhizopus spp., or Zygomycetes spp.), and phylum Oomycota in the Stramenopila kingdom.

In some embodiments, the yeast is selected from phylum Ascomycoata (e.g., Ajellomyces spp., Alternaria spp., Aschersonia spp., Aspergillus spp., Arthroderma spp., Ascochyta spp., Bipolaris spp., Blastomyces spp., Botryotinia spp., Chaetomium spp., Cladosporium spp., Coccidioides spp., Curvularia spp., Emericella spp., Emmonsia spp., Epicoccum spp., Exophiala spp., Fusarium spp., Geomyces spp., Geotrichum spp., Gibberella spp., Histoplasma spp., Magnaporthe spp., Metarhizium spp., Monascus spp., Mycospaerella spp., Nectria spp., Neosartorya spp., Neurospora spp., Paecilomyces spp., Paracoccidioides spp., Penicillium spp., Phaeosphaeria spp., Phialemonium spp., Podospora spp., Pyrenophora spp., Sclerotinia spp., Scopulariopsis spp., Sporothrix spp., Stachybotrys spp., Stemphylium spp., Talaromyces spp., Trichophyton spp., Trichothecium spp., Tricoderma spp., Tuber spp., Uncinocarpus spp., or Verticillium spp., such as Candida spp., Clavispora spp., Debaryomyces spp., Dekkera spp., Kluyveromyces spp., Pichia spp., Saccaromyces spp., Torulaspora spp., Vanderwaltozyma spp., Yarrowia spp., or Zygosaccharomyces spp.) and phylum Basidomycota (e.g., Moniliophthora spp., Sporobolomyces spp., Trichosporon spp., Ustilago spp., Cryptococcus spp., or Rhodotorula spp, such as Cryptococcus spp., or Rhodotorula spp).

In any of the embodiments described herein, the sample is selected from the group of an agricultural product (e.g., a fruit, such as a tomato, a pepper, a grape, an apple, an orange, a lemon, or a berry; a vegetable; a grain; forage; a silage; a juice; a wood; a flower; or a seed) or process, an industrial product or process, an environmental product or process, a food product or process, a pharmaceutical product or process, a water-based product or process, a wood product, a textile, and a tissue product (e.g., blood, urine, or serum).

In any of the embodiments described herein, the method includes lysing (e.g., mechanical or chemical lysis) cells present in the sample.

In any of the embodiments described herein, prior to contacting step a), the method includes denaturing complementary strands of the nucleic acid molecules.

In any of the embodiments described herein, the nucleic acid molecules include deoxyribonucleic acid molecules (DNA).

In one aspect, the invention also features a kit including (i) a first container including any mold-specific probe described herein, (ii) a second container including any yeast or mold-specific probe described herein, and (iii) instructions for performing any method described herein for detecting and discriminating between mold or yeast in a sample.

In some embodiments, the mold-specific probe and the yeast or mold-specific probe include different detectable labels (e.g., a radionuclide, a ligand (e.g., biotin or avidin), a hapten, an enzyme, an enzyme substrate, a reactive group, a chromophore (e.g., dye, particle, such as a latex or a metal particle, or bead that imparts detectable color), a luminescent compound (e.g., a bioluminescent, a phosphorescent, or a chemiluminescent label), a fluorophore, or a label, such as a homogeneous label).

In another aspect, the invention features a kit including (i) a container including any mold-specific probe described herein and any yeast or mold-specific probe described herein, where the mold-specific and yeast or mold-specific probes include different detectable labels; and (ii) instructions for performing any method described herein for detecting and discriminating between mold or yeast in a sample.

In any of the embodiments and aspects described herein, the nucleic acid molecules (e.g., probes) can include any modification described herein (e.g., a 3′ end modification to prevent initiation of DNA synthesis, such as a 3′ end methoxy modification; a 5′ end derivatization, such as a thiol group (e.g., a trityl-hexyl thiol group); a 2′ modification of the ribose, such as a methoxy modification at the 2′ position of ribose; a locked nucleic acid modification, such as a methylene bridge connecting the 2′-O atom and the 4′-C atom of ribose; a backbone modification, such as a phosphothioate modification; or a label, such as any described herein, e.g., a chemiluminescent label, a fluorophore, a hapten, an enzyme, or one of a donor/acceptor pair). In some embodiments, the nucleic acid molecules include one or more deoxyribonucleotides, ribonucleotides, peptide nucleic acids, or combinations thereof. In some embodiments, the nucleic acid molecules (e.g., probes) include SMART oligonucleotides, as described herein. In particular embodiments, the SMART oligonucleotide comprises a tag sequence at the 5′ end, where the tag sequence includes at least 70% (e.g., at least about 80%, about 85%, about 90%, about 95%, about 99%, or more, even 100%) sequence identity to a complementary sequence including at least 5 (e.g., at least 6, 7, 8, 9, 10, 11, 12, 13, 14, 15, 16, 17, 18, 19, 20, 21, 22, 23, 24, 25, 30, 35, 40, 45, 50, 55, or 60) contiguous nucleotides from the 3′ end of the tag sequence. In other embodiments, the tag sequence has a closed structure (e.g., a hairpin structure), where the 5′ end binds the 3′ end of the sequence (e.g., under stringent conditions, at an amplification temperature of at least about 40° C., such as about 42° C., or under stringent conditions at an amplification temperature of at least about 40° C., such as about 42° C.).

### DEFINITIONS

By “about” is meant +/−10% of any recited value.

By “consisting essentially of” is meant that additional component(s), composition(s), or method step(s) that do not materially change the basic and novel characteristics of the present invention may be included in the present invention. Any component(s), composition(s), or method step(s) that have a material effect on the basic and novel characteristics of the present invention would fall outside of this term.

A “label” refers to a molecular moiety or compound that is detected or leads to a detectable signal. A label may be joined directly or indirectly to a nucleic acid probe. Direct labeling can occur through bonds or interactions that link the label to the probe, including covalent bonds or non-covalent interactions, e.g. hydrogen bonds, hydrophobic and ionic interactions, or formation of chelates or coordination complexes. Indirect labeling can occur through use of a bridging moiety or linker (e.g., antibody or additional oligomer), which is either directly or indirectly labeled, and which may amplify the detectable signal. Labels include any detectable moiety, such as a radionuclide, ligand (e.g., biotin or avidin), enzyme, enzyme substrate, reactive group, chromophore (e.g., a dye, a particle, or a bead that imparts detectable color), luminescent compound (e.g., bioluminescent, phosphorescent, or chemiluminescent labels), or fluorophore. Preferred labels include a “homogeneous detectable label” that provides a detectable signal in a homogeneous fashion (i.e., without physically removing bound labels from unbound forms of the label) in which bound (e.g., hybridized) labeled probe in a mixture exhibits a detectable change that differs from that of unbound labeled probe, e.g., stability or differential degradation (e.g., U.S. Pat. No. 5,283,174, Arnold et al.; U.S. Pat. No. 5,656,207, Woodhead et al.; U.S. Pat. No. 5,658,737, Nelson et al.). Preferred labels include chemiluminescent compounds, preferably acridinium ester (“AE”) compounds that include standard AE and derivatives thereof (described in U.S. Pat. Nos. 5,656,207, 5,658,737, and 5,639,604). Methods of synthesis and attaching labels to nucleic acids and detecting signals from labels are well known (e.g., Sambrook et al., Molecular Cloning, A Laboratory Manual, 2nd ed. (Cold Spring Harbor Laboratory Press, Cold Spring Harbor, N.Y., 1989), Chpt. 10; U.S. Pat. Nos. 5,658,737, 5,656,207, 5,547,842, 5,283,174, and 4,581,333).

As used herein, a “detectable label” is a chemical species that can be detected or can lead to a detectable response. Detectable labels in accordance with the invention can be linked to polynucleotide probes either directly or indirectly, and include radioisotopes, enzymes, haptens, chromophores such as dyes or particles that impart a detectable color (e.g., latex beads or metal particles), luminescent compounds (e.g., bioluminescent, phosphorescent or chemiluminescent moieties) and fluorescent compounds. Fluorescent dyes that bind specifically to double-stranded DNA are still other examples of detectable labels.

The term “endogenous” within the context of the current invention refers to any polynucleotide, polypeptide, or protein sequence that is a natural part of a cell or organism regenerated from said cell and is not introduced to the cell or organism through human intervention or artifice.

By “endogenous chitin synthase gene” is meant a chitin synthase gene that is native to an organism and is not introduced to the organism through human intervention or artifice. Examples of endogenous chitin synthases include, but are not limited to, chitin synthases of classes I, II, III, IV, V, VI, and VII (GENBANK ACCESSION NOs: and NCBI Reference Sequence Nos. of chitin synthase class III are summarized in FIGS. 3 and 4).

By “1,3-β-glucan synthase (FKS1) gene” is meant a 1,3-β-glucan synthase gene that is native to an organism and is not introduced to the organism through human intervention or artifice (GENBANK ACCESSION NOs. and NCBI Reference Sequence NOs of 1,3-β-glucan synthase are summarized in FIGS. 6 and 7).

By “mold-specific probe” or “mold-specific primer” is meant a first nucleic acid molecule that selectively hybridizes to a second nucleic acid molecule (e.g., a gene or ribonucleic acid (RNA) product thereof) that is endogenous to a mold cell but does not selectively hybridize to a second nucleic acid molecule that is endogenous to a yeast cell.

By “mold-specific region” of a nucleic acid molecule (e.g., a gene or RNA product thereof) is meant a portion of the nucleic acid molecule (e.g., a span of about 10 base pairs to about 50 base pairs, more preferably about 10 to about 30 base pairs, and most preferably about 17 to about 20 base pairs) that selectively hybridizes to a mold-specific probe or primer but not to a yeast-specific probe or primer.

The interchangeable terms “oligo,” “oligomer,” and “oligonucleotide” refer to a nucleic acid having generally less than 1,000 nucleotides (nt), including polymers in a range having a lower limit of about 2 nt to 5 nt and an upper limit of about 500 nt to 900 nt. Preferred oligomers are in a size range having a lower limit of about 5 nt to 25 nt and an upper limit of about 50 nt to 600 nt, and particularly preferred embodiments are in a range having a lower limit of about 10 nt to 20 nt and an upper limit of about 22 nt to 100 nt. Preferred oligomers are synthesized by using any well known enzymatic or chemical method and purified by standard methods, e.g., chromatography.

By “sample” or “test sample” is meant any substance suspected of containing a target organism or nucleic acid derived from the target organism, including but not limited to plant or animal materials, waste materials, materials for forensic analysis, any tissue, cell, or extract derived from a living or dead organism which may contain a target nucleic acid. The sample may include any tissue or polynucleotide-containing material obtained from a human or animal, or it may include an environmental, laboratory-derived, or synthetic sample, a seed stock, or any other types of material where the presence of specific fungal organisms (e.g., yeast or mold) may need to be monitored. For example, samples may be obtained from environmental sources, e.g., water, soil, slurries, debris, biofilms from containers of aqueous fluids, airborne particles, or aerosols, and the like, which may include processed samples, such as those obtained from passing an environmental sample over or through a filter, by centrifugation, or by adherence to a medium, matrix, or support. “Biological samples” include any tissue or material derived from a living or dead mammal, including humans, which may contain a fungal organism or target nucleic acid derived therefrom, e.g., respiratory tissue or exudates such as bronchoscopy, bronchoalveolar lavage (BAL) or lung biopsy, sputum, peripheral blood, bone marrow, plasma, serum, lymph node, gastrointestinal tissue, urine, feces, semen, exudates, or other body fluids and the like. A sample may be treated to physically or mechanically disrupt aggregates or cells to release intracellular components, including nucleic acids, into a solution which may contain other components, such as enzymes, buffers, salts, detergents and the like.

The substance may be, for example, an unprocessed clinical specimen, a buffered medium containing the specimen, a medium containing the specimen and lytic agents for releasing nucleic acid belonging to the target organism, or a medium containing nucleic acid derived from the target organism which has been isolated and/or purified in a reaction receptacle or on a reaction material or device. In the claims, the terms “sample” and “test sample” may refer to specimen in its raw form or to any stage of processing to release, isolate, and purify nucleic acid derived from target organisms in the specimen. Thus, within a method of use claim, each reference to a “sample” or “test sample” may refer to a substance suspected of containing nucleic acid derived from the target organism or organisms at different stages of processing and is not limited to the initial form of the substance in the claim.

“Sample preparation” refers to any steps or methods that prepare a sample for subsequent amplification and detection of fungal (e.g., yeast or mold) nucleic acids present in the sample. Sample preparation may include any known method of concentrating components from a larger sample volume or from a substantially aqueous mixture, e.g., by filtration or trapping of airborne particles from an air sample or fungus from a water sample. Sample preparation may include lysis of cellular components and removal of debris, e.g., by filtration or centrifugation, and may include use of nucleic acid oligomers to selectively capture the target nucleic acid from other sample components. Sample preparation may also include “separating,” “isolating,” or “purifying,” which refers to removing one or more components from a complex mixture, such as a sample. Sample components may include target and non-target nucleic acids, and other materials such as salts, acids, bases, detergents, proteins, carbohydrates, lipids and other organic or inorganic materials. Preferably, a separating, isolating or purifying step removes at least 70%, preferably at least 90%, and more preferably about 95% of the target nucleic acids away from other sample components. A separating, isolating or purifying step may optionally include additional washing steps to remove non-target sample components.

By “selectively hybridizing” or “selective hybridization” or “selectively hybridizes” is meant hybridization, under stringent hybridization conditions, of a first nucleic acid molecule (e.g., a probe or primer) to a specified second nucleic acid molecule (e.g., a target sequence) to a detectably greater degree than its hybridization to non-target nucleic acid sequences and/or to the substantial exclusion of non-target nucleic acids. Selectively hybridizing sequences have at least 80% sequence identity, preferably 90% sequence identity, and most preferably 100% sequence identity (i.e. complementary) with each other. “Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “stringent conditions” or “stringent hybridization conditions” refer to conditions under which a probe will hybridize to its target sequence, to a detectably greater degree than other sequences. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength and pH) at which 50% of a complementary target sequence hybridizes to a perfectly matched probe. Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30% formamide, 1 M NaCl, 1% SDS at 37° C., and a wash in 2×SSC at 50° C. Exemplary high stringency conditions include hybridization in 50% formamide, 1 M NaCl, 1% SDS at 37° C., and a wash in 0.1×SSC at 60° C.

By “substantially corresponding,” or “substantially corresponds” is meant that the subject oligonucleotide has a base sequence containing an at least 10 contiguous base region (more preferably 15, 20, 25, or 30 contiguous base region) that is at least 80% homologous, preferably at least 90% homologous, and most preferably 95%, 97%, or 100% homologous to an at least 10 contiguous base region (more preferably 15, 20, 25, or 30 contiguous base region) present in a reference base sequence (excluding RNA and DNA equivalents). Those skilled in the art will readily appreciate modifications that could be made to the hybridization assay conditions at various percentages of homology to permit hybridization of the oligonucleotide to the target sequence while preventing unacceptable levels of non-specific hybridization. The degree of similarity is determined by comparing the order of nucleobases making up the two sequences and does not take into consideration other structural differences that may exist between the two sequences, provided the structural differences do not prevent hydrogen bonding with complementary bases. The degree of homology between two sequences can also be expressed in terms of the number of base mismatches present in each set of at least 10 contiguous bases (more preferably 15, 20, 25, or 30 contiguous bases) being compared, which may range from 0 to 2 base differences.

By “substantially complementary” is meant that the subject oligonucleotide has a base sequence containing an at least 10 contiguous base region (more preferably 15, 20, 25, or 30 contiguous base region) that is at least 80% complementary, preferably at least 90% complementary, and most preferably 95%, 97%, or 100% complementary to an at least 10 contiguous base region (more preferably 15, 20, 25, or 30 contiguous base region) present in a target nucleic acid sequence (excluding RNA and DNA equivalents). Those skilled in the art will readily appreciate modifications that could be made to the hybridization assay conditions at various percentages of complementarity to permit hybridization of the oligonucleotide to the target sequence while preventing unacceptable levels of non-specific hybridization. The degree of complementarity is determined by comparing the order of nucleobases making up the two sequences and does not take into consideration other structural differences which may exist between the two sequences, provided the structural differences do not prevent hydrogen bonding with complementary bases. The degree of complementarity between two sequences can also be expressed in terms of the number of base mismatches present in each set of at least 10 contiguous bases being compared, which may range from 0 to 2 base mismatches.

By “yeast or mold-specific probe,” “yeast/mold-specific probe,” “yeast or mold-specific primer,” or “yeast/mold-specific primer” is meant a first nucleic acid molecule that selectively hybridizes to a second nucleic acid molecule (e.g., a gene or ribonucleic acid (RNA) product thereof) that is endogenous to either a yeast or a mold cell or to both yeast and mold cells.

By “yeast or mold-specific region” or “yeast/mold-specific region” of a nucleic acid molecule (e.g., a gene or RNA product thereof) is meant a portion of the nucleic acid molecule (e.g., a span of about 10 base pairs to about 50 base pairs, more preferably about 10 to about 30 base pairs, and most preferably about 17 to about 20 base pairs within a gene, RNA, or cDNA) that selectively hybridizes to a yeast or mold-specific probe or primer.

Other features and advantages of the invention will be apparent from the following detailed description, the drawings, and the claims.

### BRIEF DESCRIPTION OF THE DRAWINGS

FIG. 1 is a schematic showing the phylogenetic relationships of chitin synthases in fungi.

FIG. 2 is a comparison showing a pairwise identity in a region of nucleic acid sequence of Chitin Synthase class III from different molds (SEQ ID NOs: 1-42) relative to the consensus sequence 1 (SEQ ID NO: 153).

FIG. 3 is a table showing Genbank Accession Nos. and NCBI Reference Sequence Nos. for chitin synthase class III genes from several molds (SEQ ID NOs: 1-42).

FIG. 4 is a table showing patents covering chitin synthase class III gene sequences for several molds (SEQ ID NOs: 43-64).

FIG. 5 is a comparison showing a pairwise identity in a region of nucleic acid sequence of 1,3-β-glucan synthase (FKS1) from different yeasts and molds (SEQ ID NOs: 65-125) relative to the consensus sequence 2 (SEQ ID NO: 154).

FIG. 6 is a table showing Genbank Accession Nos. and NCBI Reference Sequence Nos. for 1,3-β-glucan synthase (FKS1) genes for several yeasts and molds (SEQ ID NOs: 65-125).

FIG. 7 is a table showing patents covering 1,3-β-glucan synthase (FKS1) gene sequences for several yeasts and molds (SEQ ID NOs: 126-152).

FIG. 8 is a schematic showing the general principles of reverse transcription-mediated amplification (RTMA) according to the present invention.

FIG. 9 shows exemplary oligo sequences targeting Chitin Synthase class III for use in the RTMA methods of the present invention (SEQ ID NOs: 155-159).

FIG. 10 is a table listing the mold species that were included in an inclusivity panel for detection by the yeast/mold assay of the present invention using the Chitin Synthase class III oligo sequences of FIG. 9.

FIG. 11 is a graph showing amplification of the indicated mold species using the Chitin Synthase class III-specific oligos of FIG. 9 and 1×10⁵ copies of genomic DNA as the target input.

FIG. 12 is a graph showing amplification of the indicated mold species using the Chitin Synthase class III-specific oligos of FIG. 9 and 1×10⁴ copies of genomic DNA as the target input.

FIG. 13 is a graph showing amplification of the indicated mold species using the Chitin Synthase class III-specific oligos of FIG. 9 and 1×10³ copies of genomic DNA as the target input.

FIG. 14 is a table listing the yeast species that were included in an exclusivity panel and that were used to test mold-specific detection using the Chitin Synthase class III oligo sequences of FIG. 9.

FIG. 15 is a graph showing that DNA (1×10⁶ copies of genomic DNA as the target input) from the yeast species listed in the table of FIG. 14 was not amplified by the Chitin Synthase class III-specific oligos of FIG. 9 using the methods of the present invention. As a positive control, DNA (1×10⁵ copies of genomic DNA as the target input) from two mold species were amplified using the Chitin Synthase class III-specific oligos of FIG. 9.

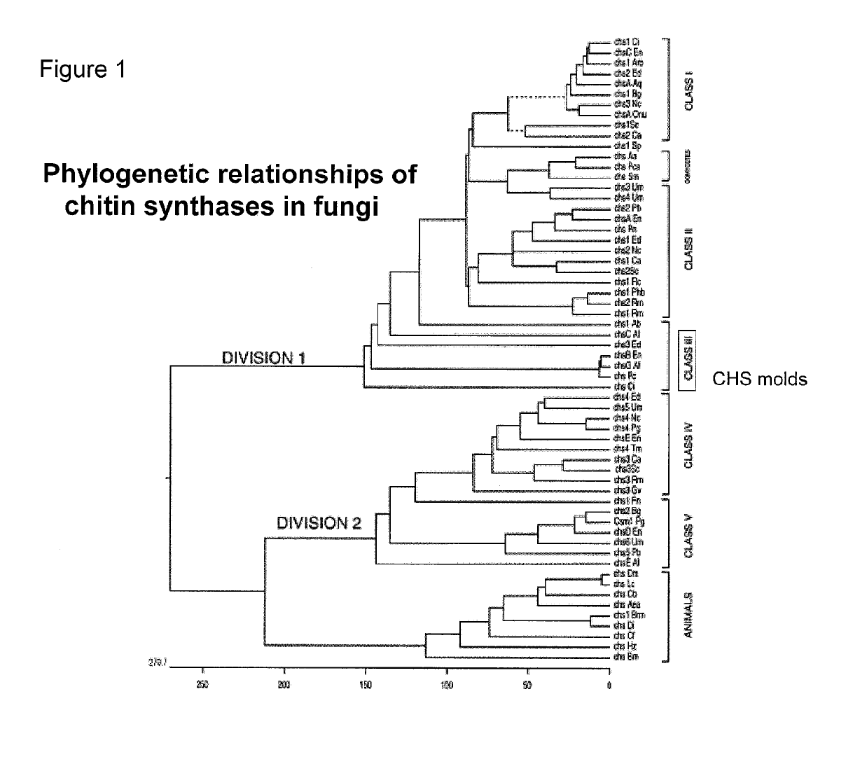

### DETAILED DESCRIPTION

The present invention relates to compositions, reaction mixtures, kits and methods for amplifying and detecting nucleic acids from various species of fungi. In particular, the compositions and methods of the invention can be used to distinguish between yeast or mold in a sample. Nucleic acid molecules obtained from a sample (e.g., tissue sample (e.g., blood), food, water, industrial, or environmental sample) can be amplified and used for nucleic acid detection, probe-based nucleic acid detection, and real-time probe-based nucleic acid detection. The sample can be a solid or semi-solid sample that is diluted with a suitable liquid (e.g., a buffer or water), or a liquid sample, such as raw and processed liquid foods, environmental samples, water, wastewater, samples taken by impingers and filtration, pharmaceuticals, and other such samples.

In certain aspects and embodiments, particular regions of chitin synthase genes or RNAs (e.g., the chitin synthase class III gene or RNA) and/or regions of 1,3-β-glucan synthase (FKS1) genes or RNAs have been identified as preferred targets for detecting at least one fungal species (mold, yeast, or both) in a sample. The target nucleic acid can be subjected to a nucleic acid amplification reaction prior to the detection step. Compositions of the current invention include one or more separate oligomers (e.g., primers and probes) useful in the amplification and detection of at least one fungal species (and preferably the oligomers allow discrimination between yeast and mold). Samples can be from any source suspected of containing a fungal species.

One object of the invention is to provide an assay procedure for rapid and easy identification of the presence of mold and/or yeast in a sample. The assay can also be used to discriminate between mold and yeast in a sample. A broad range of mold and/or yeast can be detected by the assay using, e.g., DNA or RNA probes that specifically hybridize to target sequences, e.g., sequences within one or more regions of endogenous chitin synthase genes and/or one or more regions of 1,3-β-glucan synthase (FKS1) genes. Hybridization to one or more of the target sequences allows not only detection of a fungal species (e.g., yeast or mold), but also discrimination between mold and yeast in the sample.

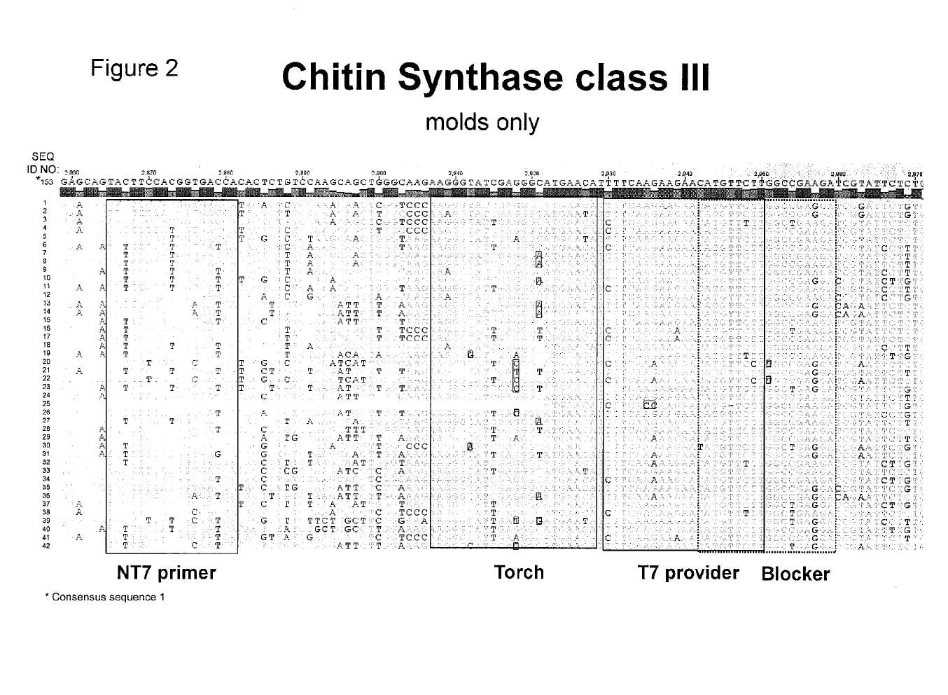

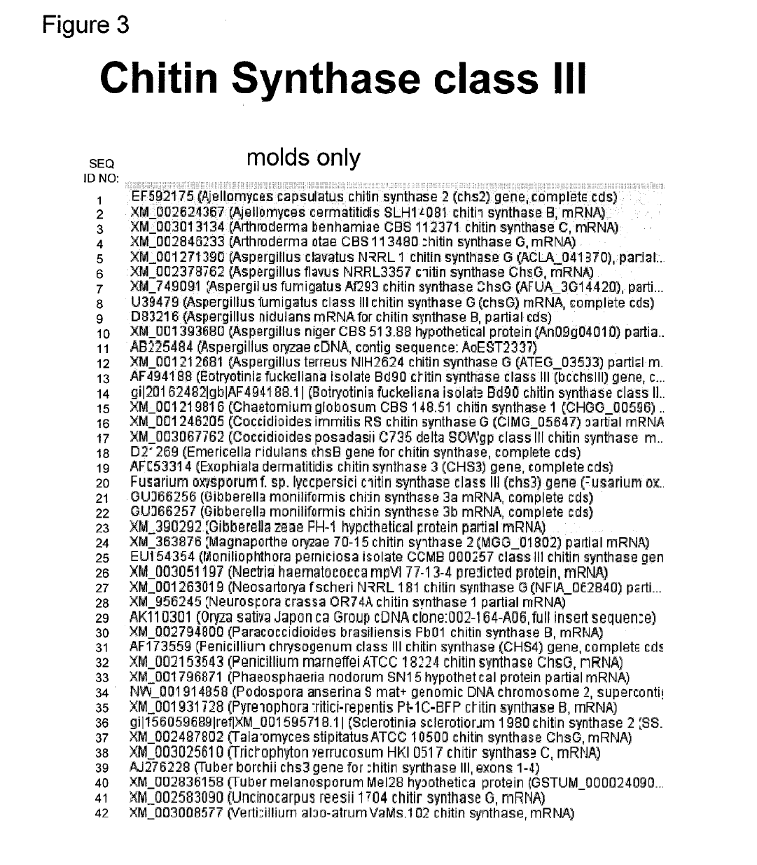

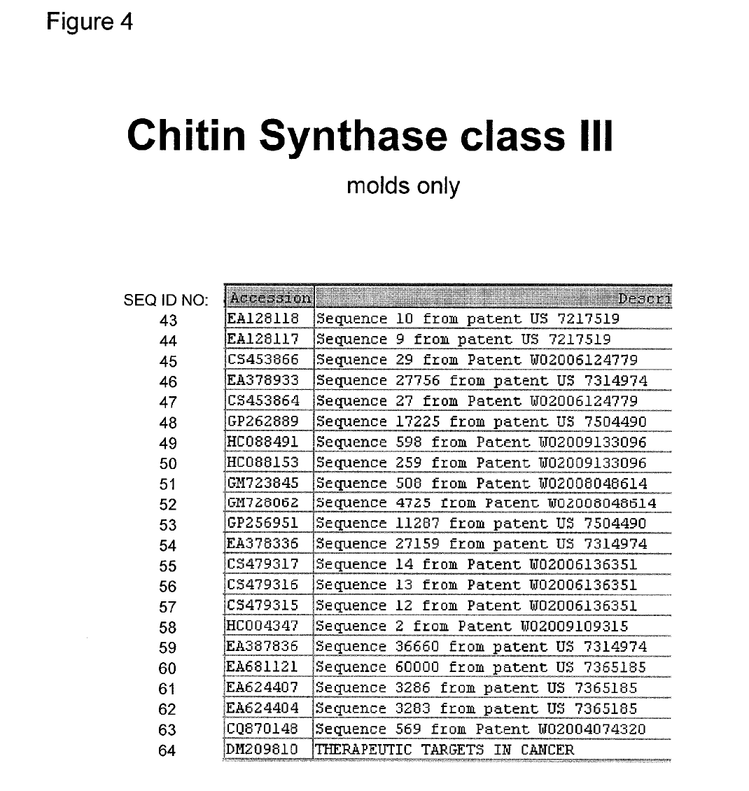

In several embodiments, the probe or primer that specifically hybridizes to an endogenous chitin synthase gene is about 17 to about 30 base pairs (e.g., at least 17, 18, 19, 20, 21, 22, 23, 24, or 25 base pairs) in length and has a sequence with at least 85% (e.g., 90%, 95%, 99%, or 100%) identity to a sequence that is complementary to a sequence of at least 17 contiguous base pairs within the region of nucleotides 2859 to 2968 shown in FIG. 2 (consensus sequence 1 (SEQ ID NO: 153); e.g., the probe or primer has a sequence with at least 85% (e.g., 90%, 95%, 99%, or 100%) identity to a sequence that is complementary to a sequence of at least 17 contiguous base pairs within one or more of the sequences of SEQ ID NOs: 1-42 that are shown in the alignment in FIG. 2). In yet other embodiments, the endogenous chitin synthase gene probe or primer has a sequence that is complementary to a sequence of at least 17 contiguous base pairs within one or more of SEQ ID NOs: 1-42 that shares at least 70% (more preferably 80%, 85%, 90%, 95%, or 99% or more) sequence identity to the region within nucleotides 2859 to 2968 shown in FIG. 2 (consensus sequence 1; SEQ ID NO: 153) between, e.g., at least 3, 5, 10, 15, or 20 or more fungal species (e.g., up to 50 or more species).

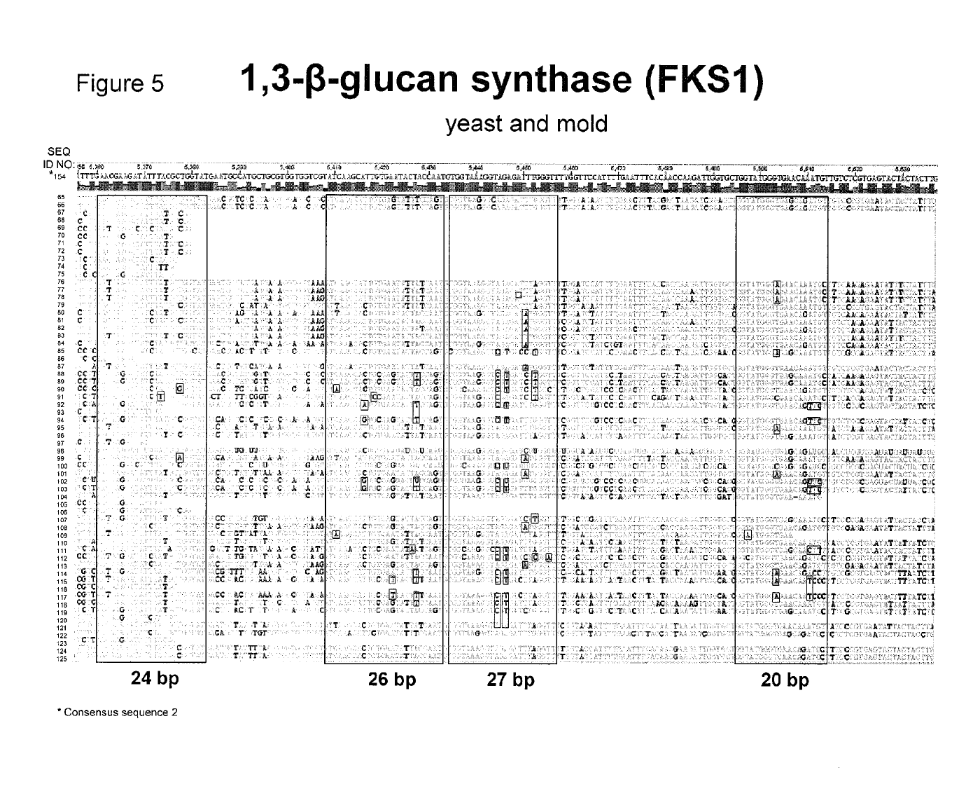

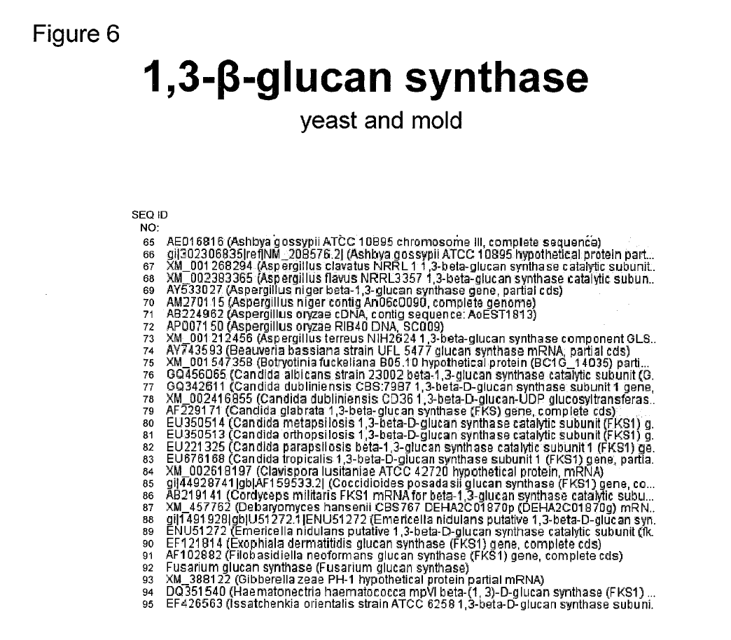

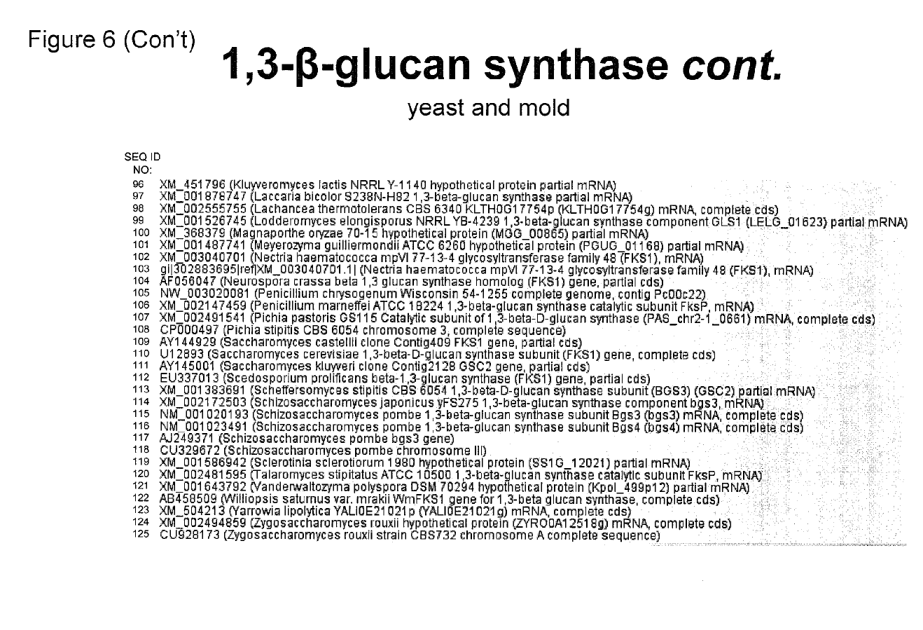

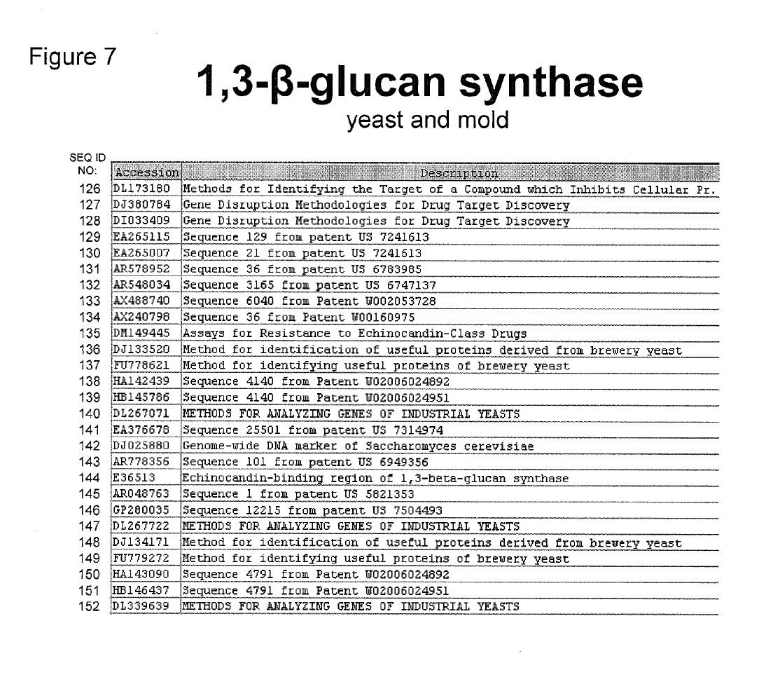

In several embodiments, the probe or primer that specifically hybridizes to an endogenous 1,3-β-glucan synthase (FKS1) gene is about 17 to about 30 base pairs (e.g., at least 17, 18, 19, 20, 21, 22, 23, 24, or 25 base pairs) in length and has a sequence with at least 85% (e.g., 90%, 95%, 99%, or 100%) identity to a sequence that is complementary to a sequence of at least 17 contiguous base pairs within the region of nucleotides 5360 to 5515 shown in FIG. 5 (consensus sequence 1 (SEQ ID NO: 154); e.g., the probe or primer has a sequence with at least 85% (e.g., 90%, 95%, 99%, or 100%) identity to a sequence that is complementary to a sequence of at least 17 contiguous base pairs within one or more of the sequences of SEQ ID NOs: 65-125 that are shown in the alignment in FIG. 5). In yet other embodiments, the 1,3-β-glucan synthase (FKS1) gene probe or primer has a sequence that is complementary to a sequence of at least 17 contiguous base pairs within one or more of SEQ ID NOs: 65-125 that shares at least 70% (more preferably 80%, 85%, 90%, 95%, or 99% or more) sequence identity to the region within nucleotides 5360 to 5515 shown in FIG. 5 (consensus sequence 2; SEQ ID NO: 154) between, e.g., at least 3, 5, 10, 15, or 20 or more fungal species (e.g., up to 50 or more species).

The assay of the invention can be used to detect various molds, including but not limited molds from phylum Ascomycota (e.g., Ajellomyces spp., Alternaria spp., Aschersonia spp., Aspergillus spp., Arthroderma spp., Ascochyta spp., Bipolaris spp., Blastomyces spp., Byssochlamys spp., Botryotinia spp., Chaetomium spp., Cladosporium spp., Coccidioides spp., Curvularia spp., Emericella spp., Emmonsia spp., Epicoccum spp., Exophiala spp., Fusarium spp., Geotrichum spp., Gibberella spp., Histoplasma spp., Magnaporthe spp., Metarhizium spp., Monascus spp., Mycospaerella spp., Nectria spp., Neosartorya spp., Neurospora spp., Paecilomyces spp., Paracoccidioides spp., Penicillium spp., Phaeosphaeria spp., Phialemonium spp., Podospora spp., Pyrenophora spp., Sclerotinia spp., Scopulariopsis spp., Sporothrix spp., Stachybotrys spp., Stemphylium spp., Talaromyces spp., Trichophyton spp., Trichothecium spp., Tricoderma spp., Tuber spp., Uncinocarpus spp., or Verticillium spp.), phylum Basidomycota (e.g., Moniliophthora spp., Sporobolomyces spp., Trichosporon spp., Ustilago spp., Cryptococcus spp. or Rhodotorula spp.), phylum Chytridiomycota, phylum Zygomycota (e.g., Absidia spp., Amylomyces spp., Pilaira spp., Rhizomucor spp., Rhizopus spp., or Zygomycetes spp.), and phylum Oomycota in the Stramenopila kingdom.

The assay of the invention can be used to detect various yeasts, including but not limited yeasts from phylum Ascomycoata (e.g., Candida spp., Clavispora spp., Debaryomyces spp., Dekkera spp., Kluyveromyces spp., Pichia spp., Saccaromyces spp., Torulaspora spp., Vanderwaltozyma spp., Yarrowia spp., or Zygosaccharomyces spp.) and phylum Basidomycota (e.g., Cryptococcus spp. or Rhodotorula spp.).

In other embodiments, the probes and primers of the invention are directed to one or more regions within a chitin synthase gene or RNA (e.g., chitin synthase class I-VII) and/or one or more regions within a 1,3-β-glucan synthase (FKS1) gene or RNA. Preferably, the probes and primers are directed to one or more regions within a chitin synthase class III gene or RNA. The primers of the invention can be used for PCR amplification, while the probes can be used for detection of, and discrimination between, yeast or mold (or yeast and mold) in a sample.

Probes and primers directed against chitin synthase genes or RNAs or 1,3-β-glucan synthase (FKS1) genes or RNAs are selected for the following reasons: Firstly, the chitin synthase gene products and the 1,3-β-glucan synthase (FKS1) gene products are clinically relevant, as they are the enzymes responsible for synthesizing chitin and 1,3-β-glucan, respectively, the major cell wall components found in fungi. The enzyme chitin synthase is upregulated in the filamentous pseudohyphae, the structure involved in tissue invasion. Secondly, the chitin synthase and 1,3-β-glucan synthase (FKS1) genes are fungal-specific, having no mammalian, bacterial, or viral counterparts. This is an important point in avoiding false positive amplification of mammalian DNA which will be present in clinical or other samples.

For example, patients' blood samples contain nucleated cells containing the patients' own DNA and RNA. Thus, this DNA and/or RNA will be co-extracted with fungal DNA and/or RNA during sample preparation for analysis. Consequently, one does not want to amplify a fungal gene that has a mammalian homolog since one cannot separate mammalian DNA from the target fungal DNA.

The nucleic acid targets for the yeast and mold assay described herein include, e.g., single copy genes, such as a chitin synthase gene (e.g., chitin synthase class III) or 1,3-β-glucan synthase (FKS1) gene. Preferably, the target nucleic acid is fungi-specific or has low homology to nucleic acid molecules (genes or RNAs) from other eukaryotes. In addition, the target nucleic acid should be one that is highly conserved between multiple fungal species (e.g., having at least 70% sequence similarity (or at least 75%, 80%, 85%, 90%, 95%, 97%, 99%, or 100% sequence similarity) between 2 or more fungal species (e.g., at least 3, 5, 10, 15, or 20 or more fungal species, e.g., up to 50 or more species).

Strategies for identifying fungal-specific single copy genes include, e.g., performing a literature search for fungal single-copy gene targets, sequence BLAST searches, and/or sequence alignments using Geneious bioinformatics software (Biomatters Ltd, Auckland, New Zealand). To avoid false positive results, the nucleic acid probes and primers to be used in the yeast and mold-specific assay of the present invention can be selected from those that are directed to fungal-specific single-copy targets in order to avoid homology to other eukaryotes. Furthermore, the fungal-specific target genes or RNAs preferably have amplifiable regions (e.g., conserved 20-60 base pair regions) for use in amplification techniques. The amplifiable regions are preferably ones that can be used to differentiate between yeasts and molds. The target genes may also include an additional 10-40 base pair region (e.g., a 20 base pair region) within a 100 base pair distance downstream from the amplifiable region that can be used to design a complementary sequence referred to as a blocker. As is discussed below, the blocker can be used in a TMA/RTMA-based assay to reduce the amount of non-target nucleic acid molecules present in a sample.

Preferably, the yeast/mold assay is based on a nucleic acid amplification reaction, but other assays that do not amplify the target nucleic acid molecules (i.e., direct detection) can also be used. These assays include, e.g., immunoassays, bio-sensor assays, immunostaining-microscopy-based assays, nucleic acid-array-based assays, DNA chip-based assays, bacteriophage-detection-based assays, classical microbiology-based assays, and chemical or biochemical assays based on the detection of compounds associated with particular target organisms or groups of target organisms, and combinations thereof.

In other embodiments, the yeast/mold assay is performed using a liquid-based assay or using a solid-support based assay (see, e.g., U.S. Pat. No. 7,745,119).

### Useful Probe Labeling Systems and Detectable Moieties

Preferred detectable labels for probes in accordance with the present invention are detectable in homogeneous assay systems (i.e., where, in a mixture, bound labeled probe exhibits a detectable change, such as stability or differential degradation, compared to unbound labeled probe).

Essentially any labeling and detection system that can be used for monitoring specific nucleic acid hybridization can be used in conjunction with the present invention. Included among the collection of useful labels are fluorescent moieties (either alone or in combination with “quencher” moieties), chemiluminescent molecules, and redox-active moieties that are amenable to electronic detection methods. In some embodiments, preferred fluorescent labels include non-covalently binding labels (e.g., intercalating dyes) such as ethidium bromide, propidium bromide, chromomycin, acridine orange, and the like. In other embodiments, the use of covalently bound fluorescent agents is preferred. Preferred chemiluminescent molecules include acridinium esters. Examples of these detectable labels have been disclosed by Arnold et al., in U.S. Pat. No. 5,283,174 for use in connection with homogenous protection assays. Preferred electronic labeling and detection approaches are disclosed in U.S. Pat. Nos. 5,591,578 and 5,770,369, and the published international patent application WO 98/57158. Redox active moieties useful as labels in the present invention include transition metals, such as Cd, Mg, Cu, Co, Pd, Zn, Fe, and Ru.

In some applications, probes exhibiting at least some degree of self-complementarity are desirable to facilitate detection of probe:target duplexes in a test sample without first requiring the removal of unhybridized probe prior to detection. By way of example, structures referred to as “molecular torches” and “molecular beacons” are designed to include distinct regions of self-complementarity and regions of target-complementarity. Molecular torches are fully described in U.S. Pat. Nos. 6,849,412, 6,835,542, 6,534,274, and 6,361,945, and molecular beacons are fully described in U.S. Pat. Nos. 5,118,801, 5,312,728, and 5,925,517. Both of these self-reporting probes include a label pair that interacts when the probe is in a closed conformation in the absence of any hybridized target. Hybridization of the target nucleic acid and the target complementary sequence separates the self-complementary portions of the probes, thereby shifting the probes to an open conformation. The shift to the open conformation is detectable due to reduced interaction of the label pair, which may be, for example, a fluorophore and a quencher (e.g., DABCYL and EDANS). Examples of other forms of labeled probes are disclosed in, e.g., U.S. Pat. Nos. 5,118,801, 5,312,728, and 6,150,097, and U.S. Pub. Nos. 2006-0068417 A1 and 2006-0194240 A1.

In preferred embodiments that detect an amplified product near or at the end of an amplification step, the molecular torch, as a linear probe, hybridizes to the amplified product to provide a signal that indicates hybridization of the probe to the amplified sequence. In preferred embodiments that use real-time detection, the probe is preferably a hairpin structure probe that includes a reporter moiety that provides the detected signal when the probe binds to the amplified product. For example, a hairpin probe may include a reporter moiety or label, such as a fluorophore (“F”), attached to one end of the probe and an interacting compound, such as quencher (“Q”), attached to the other end the hairpin structure to inhibit signal production when the hairpin structure is in the “closed” conformation and not hybridized to the amplified product, whereas a detectable signal results when the probe is hybridized to a complementary sequence in the amplified product, thus converting the probe to a “open” conformation.

Molecular torches, molecular beacons and the like preferably are individually labeled with an interactive pair of detectable labels. Examples of detectable labels that are preferred as members of an interactive pair of labels interact with each other by FRET or non-FRET energy transfer mechanisms. Fluorescence resonance energy transfer (FRET) involves the radiationless transmission of energy quanta from the site of absorption to the site of its utilization in the molecule, or system of molecules, by resonance interaction between chromophores, over distances considerably greater than interatomic distances, without conversion to thermal energy, and without the donor and acceptor coming into kinetic collision. The “donor” is the moiety that initially absorbs the energy, and the “acceptor” is the moiety to which the energy is subsequently transferred. In addition to FRET, there are at least three other “non-FRET” energy transfer processes by which excitation energy can be transferred from a donor to an acceptor molecule.

Examples of donor/acceptor label pairs that may be used in connection with the invention, making no attempt to distinguish FRET from non-FRET pairs, include fluorescein/tetramethylrhodamine, IAEDANS/fluorescein, EDANS/DABCYL, coumarin/DABCYL, fluorescein/fluorescein, BODIPY FL/BODIPY FL, fluorescein/DABCYL, lucifer yellow/DABCYL, BODIPY/DABCYL, eosin/DABCYL, erythrosine/DABCYL, tetramethylrhodamine/DABCYL, Texas Red/DAB CYL, CY5/BH1, CY5/BH2, CY3/BH1, CY3/BH2 and fluorescein/QSY7 dye. Those having an ordinary level of skill in the art will understand that when donor and acceptor dyes are different, energy transfer can be detected by the appearance of sensitized fluorescence of the acceptor or by quenching of donor fluorescence. When the donor and acceptor species are the same, energy can be detected by the resulting fluorescence depolarization. Non-fluorescent acceptors such as DABCYL and the QSY 7 dyes advantageously eliminate the potential problem of background fluorescence resulting from direct (i.e., non-sensitized) acceptor excitation. Preferred fluorophore moieties that can be used as one member of a donor-acceptor pair include fluorescein, ROX, TAMARA, and the CY dyes (such as CY5). Highly preferred quencher moieties that can be used as another member of a donor-acceptor pair include DABCYL and the BLACK HOLE QUENCHER moieties which are available from Biosearch Technologies, Inc., (Novato, Calif.).

Certain amplicon monitoring procedures most commonly used in connection with PCR-based methods rely on the use of DNA binding dyes or probes which can be digested by a nuclease activity. In the first instance, the amount of analyte amplicon present in a reaction preferably is monitored using fluorescent double-stranded DNA recognizing compounds. This is possible because the amount of double-stranded amplification product usually exceeds the amount of nucleic acid originally present in the sample to be analyzed. Double-stranded DNA specific dyes may be used in these monitoring procedures so that, upon excitation with an appropriate wavelength of light, enhanced fluorescence results only if the dye is bound to double-stranded DNA. Preferably, only those dyes are used which, like SYBR Green I (Molecular Probes/Invitrogen Corporation; CA), do not affect the efficiency of the PCR reaction. In an alternative procedure, the amplification product is detected using a single-stranded hybridization probe which is labeled with a fluorescent entity. The fluorescence emission of this entity is quenched by a second label which is present on the same probe, and which acts as a quenching compound. During the annealing step of the PCR reaction, the probe hybridizes to its target sequence, and, subsequently, during the extension of the primer, the DNA polymerase having a 5′-3′-exonuclease activity digests the hybridization probe such that the fluorescent entity is separated from the quencher compound. After appropriate excitation, fluorescence emission can be monitored as an indicator of accumulating amplification product.

Synthetic techniques and methods of bonding labels to nucleic acids and detecting labels are well known in the art (e.g., see Sambrook et al., Molecular Cloning, A Laboratory Manual, 2nd ed. (Cold Spring Harbor Laboratory Press, Cold Spring Harbor, N.Y., 1989), Chapter 10; Nelson et al., U.S. Pat. No. 5,658,737; Woodhead et al., U.S. Pat. No. 5,656,207; Hogan et al., U.S. Pat. No. 5,547,842; Arnold et al., U.S. Pat. No. 5,283,174; Kourilsky et al., U.S. Pat. No. 4,581,333), and Becker et al., European Patent App. No. 0 747 706.

### Yeast/Mold Assay

The present invention provides a method for detecting and distinguishing between yeast and/or mold using a hybridization assay that may also include a nucleic amplification step that precedes a hybridization step. Preparation of samples for amplification of yeast and/or mold sequences may include separating and/or concentrating organisms contained in a sample from other sample components, e.g., filtration of particulate matter from air, water, or other types of samples. Once separated or concentrated, the target nucleic acid may be obtained from any medium of interest, for example, a liquid sample of medical, veterinary, environmental, or industrial significance. Sample preparation may also include chemical, mechanical, and/or enzymatic disruption of cells to release intracellular contents, including yeast and/or mold RNA or DNA. Preferred samples are food and environmental samples.

Sample preparation may include a step of target capture to specifically or non-specifically separate the target nucleic acids from other sample components. Nonspecific target preparation methods may selectively precipitate nucleic acids from a substantially aqueous mixture, adhere nucleic acids to a support that is washed to remove other sample components, or use other means to physically separate nucleic acids, including yeast and/or mold nucleic acid, from a mixture that contains other components. Other nonspecific target preparation methods may selectively separate RNA from DNA in a sample.

The target nucleic acid may also be the product of a nucleic acid amplification assay, such as PCR or ligase chain reaction (LCR). If the target nucleic acid is principally double stranded, it will be treated to denature it prior to the hybridization assay. Denaturation of nucleic acids is preferably accomplished by heating in boiling water or alkali treatment (e.g., 0.1 N sodium hydroxide), which, if desired, can simultaneously be used to lyse cells. Also, release of nucleic acids from cellular sources can, for example, be obtained by mechanical disruption (freeze/thaw, abrasion, sonication, bead beating), physical/chemical disruption (detergents such as TRITON™, Tween, or sodium dodecyl sulfate, alkali treatment, osmotic shock, or heat), or enzymatic lysis (lysozyme, proteinase K, pepsin). The resulting test medium will contain the target nucleic acid in single-stranded form.

Reaction mixtures for amplifying the target nucleic acids may include one or more of target capture reagents, blocker nucleic acid molecules, lysis reagents, and amplification reagents. All or some of the oligomer compositions may be present in one or more of such reaction mixtures. The methods may include performing a nucleic acid amplification of fungal sequences (e.g., chitin synthase (mold only) or 1,3-β-glucan synthase (FKS1) (yeast and mold) gene or RNA sequences). Amplified nucleic acids are then useful for a variety of subsequent analytical methods, including, but not limited to nucleic acid detection, for example by specifically hybridizing the amplified product with a nucleic acid probe that provides a signal to indicate the presence of a mold or yeast in the sample. The amplification step includes contacting the sample with one or more amplification oligomers specific for a target sequence in, e.g., a chitin synthase gene or RNA (e.g., a chitin synthase class III gene or RNA) or a 1,3-β-glucan synthase (FKS1) gene or RNA to produce an amplified product if a fungal nucleic acid is present in the sample. Amplification synthesizes additional copies of the target sequence or its complement by using at least one nucleic acid polymerase to extend the sequence from an amplification oligomer (a primer) using a template strand. One embodiment for detecting the amplified product uses a hybridizing step that includes contacting the amplified product with at least one probe specific for a sequence amplified by the selected amplification oligomers, e.g., a sequence contained in the target sequence flanked by a pair of selected primers (e.g., a target sequence present within a chitin synthase gene or RNA, such as a chitin synthase class III gene or RNA, or a 1,3-β-glucan synthase (FKS1) gene or RNA).

Amplification methods known in the art include, for example, replicase-mediated amplification, polymerase chain reaction (PCR), ligase chain reaction (LCR), strand-displacement amplification (SDA), and transcription-mediated or transcription-associated amplification (see, e.g., U.S. Pat. No. 6,872,816). Replicase-mediated amplification uses self-replicating RNA molecules, and a replicase such as QB-replicase (see, e.g., U.S. Pat. No. 4,786,600). PCR amplification uses a DNA polymerase, pairs of primers, and thermal cycling to synthesize multiple copies of two complementary strands of dsDNA or from a cDNA (see, e.g., U.S. Pat. Nos. 4,683,195; 4,683,202; and 4,800,159). LCR amplification uses four or more different oligonucleotides to amplify a target and its complementary strand by using multiple cycles of hybridization, ligation, and denaturation (see, e.g., U.S. Pat. Nos. 5,427,930 and 5,516,663). SDA uses a primer that contains a recognition site for a restriction endonuclease and an endonuclease that nicks one strand of a hemimodified DNA duplex that includes the target sequence, whereby amplification occurs in a series of primer extension and strand displacement steps (see, e.g., U.S. Pat. Nos. 5,422,252; 5,547,861; and 5,648,211). Those skilled in the art will know how to provide suitable conditions for performing these amplification reactions.

The target nucleic acid of one or more fungal species (e.g., mold and/or yeast) can also be amplified by a transcription-based amplification technique. One transcription-based amplification system is transcription-mediated amplification (TMA), which employs an RNA polymerase to produce multiple RNA transcripts of a target region. Exemplary TMA amplification methods are described in, e.g., U.S. Pat. Nos. 4,868,105; 5,124,246; 5,130,238; 5,399,491; 5,437,990; 5,480,784; 5,554,516; and 7,374,885; and PCT Pub. Nos. WO 88/01302; WO 88/10315 and WO 95/03430.

The methods of the present invention may include a TMA reaction that involves the use of a single primer TMA reaction, as is described in U.S. Pat. No. 7,374,885. In general, the single-primer TMA methods use a primer oligomer (e.g., a NT7 primer), a modified promoter-based oligomer (or “promoter-provider oligomer”; e.g., a T7 provider) that is modified to prevent the initiation of DNA synthesis from its 3′ end (e.g., by including a 3′-blocking moiety) and, optionally, a blocker oligomer (e.g., a blocker) to terminate elongation of a cDNA from the target strand (see, e.g., FIG. 2). Promoter-based oligomers provide an oligonucleotide sequence that is recognized by an RNA polymerase. This single primer TMA method synthesizes multiple copies of a target sequence and includes the steps of treating a target RNA that contains a target sequence with a priming oligomer and a binding molecule, where the primer hybridizes to the 3′ end of the target strand. RT initiates primer extension from the 3′ end of the primer to produce a cDNA which is in a duplex with the target strand (e.g., RNA:cDNA). When a blocker oligomer, is used in the reaction, it binds to the target nucleic acid adjacent near the user designated 5′ end of the target sequence. When the primer is extended by DNA polymerase activity of RT to produce cDNA, the 3′ end of the cDNA is determined by the position of the blocker oligomer because polymerization stops when the primer extension product reaches the binding molecule bound to the target strand. Thus, the 3′ end of the cDNA is complementary to the 5′ end of the target sequence. The RNA:cDNA duplex is separated when RNase (e.g., RNase H of RT) degrades the RNA strand, although those skilled in the art will appreciate that any form of strand separation may be used. Then, the promoter-provider oligomer hybridizes to the cDNA near the 3′ end of the cDNA strand.

The promoter-provider oligomer includes a 5′ promoter sequence for an RNA polymerase and a 3′ target hybridizing region complementary to a sequence in the 3′ region of the cDNA. The promoter-provider oligomer also has a modified 3′ end that includes a blocking moiety that prevents initiation of DNA synthesis from the 3′ end of the promoter-provider oligomer. In the promoter-provider:cDNA duplex, the 3′-end of the cDNA is extended by DNA polymerase activity of RT using the promoter oligomer as a template to add a promoter sequence to the cDNA and create a functional double-stranded promoter.

An RNA polymerase specific for the promoter sequence then binds to the functional promoter and transcribes multiple RNA transcripts complementary to the cDNA and substantially identical to the target region sequence that was amplified from the initial target strand. The resulting amplified RNA can then cycle through the process again by binding the primer and serving as a template for further cDNA production, ultimately producing many amplicons from the initial target nucleic acid present in the sample. Some embodiments of the single-primer transcription-associated amplification method do not include the blocking oligomer and, therefore, the cDNA product made from the primer has an indeterminate 3′ end, but the amplification steps proceed substantially as described above for all other steps.

The methods of the invention may also utilize a reverse transcription-mediated amplification (RTMA), various aspects of which are disclosed in, e.g., U.S. Pat. Pub. No. US 2006-0046265 A1. RTMA is an RNA transcription-mediated amplification system using two enzymes to drive the reaction: RNA polymerase and reverse transcriptase. RTMA is isothermal; the entire reaction is performed at the same temperature in a water bath or heat block. This is in contrast to other amplification reactions such as PCR that require a thermal cycler instrument to rapidly change the temperature to drive reaction. RTMA can amplify either DNA or RNA, and can produce either DNA or RNA amplicons, in contrast to most other nucleic acid amplification methods that only produce DNA. RTMA has very rapid kinetics, resulting in a billion-fold amplification within 15-60 minutes. RTMA can be combined with a Hybridization Protection Assay (HPA), which uses a specific oligonucleotide probe labeled with an acridinium ester detector molecule that emits a chemiluminescent signal, for endpoint detection or with molecular torches for real-time detection. There are no wash steps, and no amplicon is ever transferred out of the tube, which simplifies the procedure and reduces the potential for contamination. Thus, the advantages of RTMA include amplification of multiple targets, results can be qualitative or quantitative, no transfers and no wash steps necessary, and detection can be in real time using molecular torches.

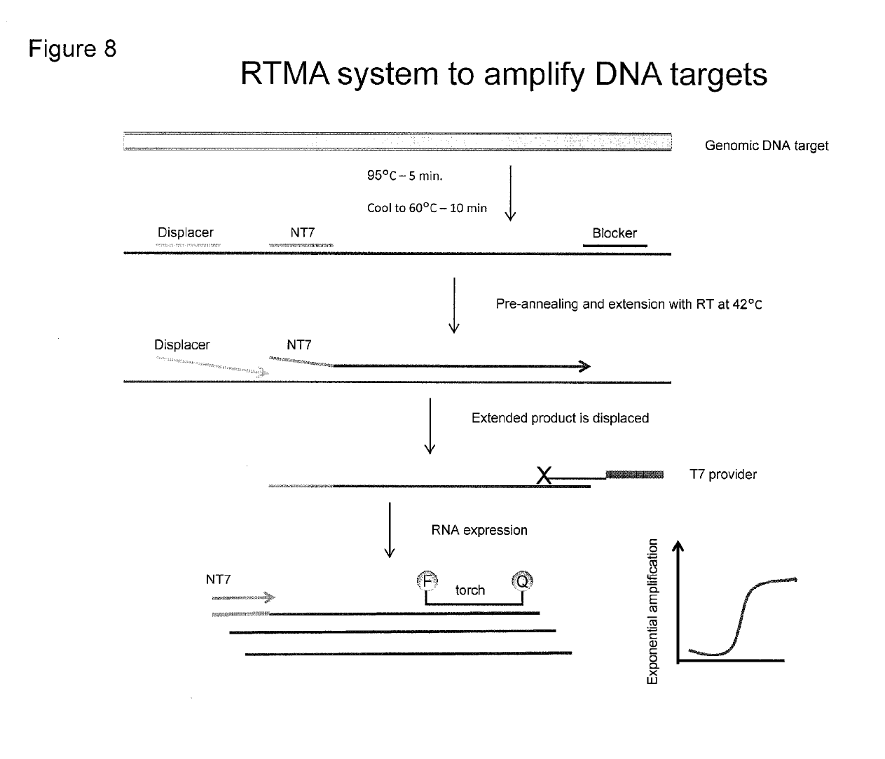

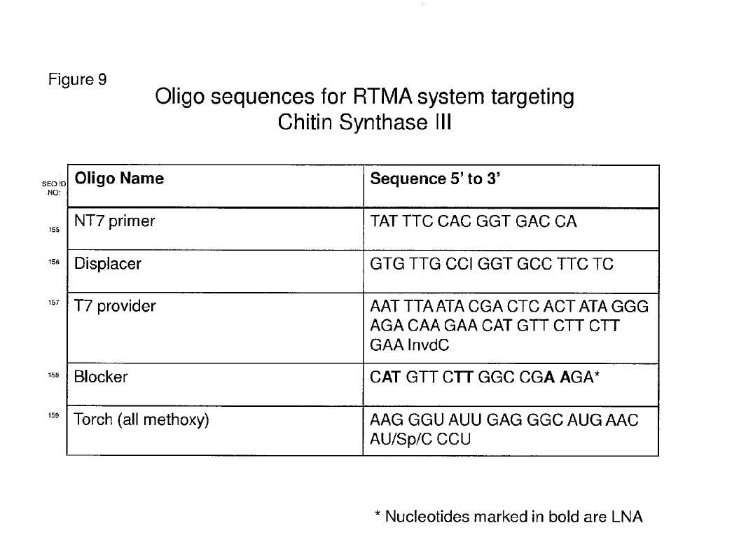

As an illustrative embodiment, the RTMA reaction is initiated by treating an RNA target sequence in a nucleic acid sample with both a tagged amplification oligomer and, optionally a blocking oligomer (see, e.g., FIG. 8). The tagged amplification oligomer includes a target hybridizing region that hybridizes to a 3′-end of the target sequence and a tag region situated 5′ to the target hybridizing region. The blocking oligomer hybridizes to a target nucleic acid containing the target sequence in the vicinity of the 5′-end of the target sequence. Thus, the target nucleic acid forms a stable complex with the tagged amplification oligomer at the 3′-end of the target sequence and the terminating oligonucleotide located adjacent to or near the determined 5′-end of the target sequence prior to initiating a primer extension reaction. Unhybridized tagged amplification oligomers are then made unavailable for hybridization to a target sequence prior to initiating a primer extension reaction with the tagged priming oligonucleotide, preferably by inactivating and/or removing the unhybridized tagged priming oligonucleotide from the nucleic acid sample. Unhybridized tagged amplification oligomer that has been inactivated or removed from the system is then unavailable for unwanted hybridization to contaminating nucleic acids. In one example of removing unhybridized tagged amplification oligomer from a reaction mixture, the tagged amplification oligomer is hybridized to the target nucleic acid, and the tagged amplification oligomer:target nucleic acid complex is removed from the unhybridized tagged amplification oligomer using a wash step. In this example, the tagged amplification oligomer:target nucleic acid complex may be further complexed to a target capture oligomer and a solid support. In one example of inactivating the unhybridized tagged amplification oligomer, the tagged amplification oligomers further comprise a target-closing region. In this example, the target hybridizing region of the tagged amplification oligomer hybridizes to target nucleic acid under a first set of conditions (e.g., stringency). Following the formation of the tagged amplification oligomer:target nucleic acid complex the unhybridized tagged amplification oligomer is inactivated under a second set of the conditions, thereby hybridizing the target closing region to the target hybridizing region of the unhybridized tagged amplification oligomer. The inactivated tagged amplification oligomer is then unavailable for hybridizing to contaminating nucleic acids. A wash step may also be included to remove the inactivated tagged amplification oligomers from the assay.

An extension reaction is then initiated from the 3′-end of the tagged amplification oligomer with a DNA polymerase, e.g., reverse transcriptase, to produce an initial amplification product that includes the tag sequence. The initial amplification product is then separated from the target sequence using an enzyme that selectively degrades the target sequence (e.g., RNAse H activity). Next, the initial amplification product is treated with a promoter-based oligomer having a target hybridizing region and an RNA polymerase promoter region situated 5′ to the target hybridizing region, thereby forming a promoter-based oligomer:initial amplification product hybrid. The promoter-based oligomer may be modified to prevent the initiation of DNA synthesis, preferably by situating a blocking moiety at the 3′-end of the promoter-based oligomer (e.g., nucleotide sequence having a 3′-to-5′ orientation). The 3′-end of the initial amplification product is then extended to add a sequence complementary to the promoter, resulting in the formation of a double-stranded promoter sequence. Multiple copies of a RNA product complementary to at least a portion of the initial amplification product are then transcribed using an RNA polymerase, which recognizes the double-stranded promoter and initiates transcription therefrom. As a result, the nucleotide sequence of the RNA product is substantially identical to the nucleotide sequence of the target nucleic acid and to the complement of the nucleotide sequence of the tag sequence.

The RNA products may then be treated with a tag-targeting oligomer, which hybridizes to the complement of the tag sequence to form a tag-targeting oligomer: RNA product hybrid, and the 3′-end of the tag-targeting oligomer is extended with the DNA polymerase to produce an amplification product complementary to the RNA product. The DNA strand of this amplification product is then separated from the RNA strand of this amplification product using an enzyme that selectively degrades the first RNA product (e.g., RNAse H activity). The DNA strand of the amplification product is treated with the promoter-based oligomer, which hybridizes to the 3′-end of the second DNA primer extension product to form a promoter-based oligomer:amplification product hybrid. The promoter-based oligomer:amplification product hybrid then re-enters the amplification cycle, where transcription is initiated from the double-stranded promoter and the cycle continues, thereby providing amplification product of the target sequence.

Amplification product can then be used in a subsequent assay. One subsequent assay includes nucleic acid detection, preferably nucleic acid probe-based nucleic acid detection. The detection step may be performed using any of a variety of known ways to detect a signal specifically associated with the amplified target sequence, such as by hybridizing the amplification product with a labeled probe and detecting a signal resulting from the labeled probe. The detection step may also provide additional information on the amplified sequence, such as all or a portion of its nucleic acid base sequence. Detection may be performed after the amplification reaction is completed, or may be performed simultaneous with amplifying the target region, e.g., in real time. In one embodiment, the detection step allows detection of the hybridized probe without removal of unhybridized probe from the mixture (see, e.g., U.S. Pat. Nos. 5,639,604 and 5,283,174).

In embodiments that detect the amplified product near or at the end of the amplification step, a linear probe may be used to provide a signal to indicate hybridization of the probe to the amplified product. One example of such detection uses a probe that is labeled with a luminescent marker. The luminescent label may be hydrolyzed from non-hybridized probe. Detection is performed by chemiluminescence using a luminometer (see, e.g., WO 89/002476).

In other embodiments that use real-time detection, the probe may be a hairpin probe, such as a molecular beacon, molecular torch, or hybridization switch probe that is labeled with a reporter moiety that is detected when the probe binds to amplified product. Such probes may contain target binding sequences and non-target binding sequences. Various forms of such probes have been described previously (see, e.g., U.S. Pat. Nos. 5,118,801; 5,312,728; 5,925,517; 6,150,097; 6,849,412; 6,835,542; 6,534,274; and 6,361,945; and U.S. Pat. Pub. Nos. 2006-0068417A1 and 2006-0194240A1).

The nucleic acid probe used to detect the target nucleic acid may be a DNA probe, an RNA probe, or a peptide nucleic acid (PNA) probe. The nucleic acid probe will have at least one single-stranded base sequence substantially complementary to at least part of the target nucleic acid sequence. However, such base sequence need not be a single continuous polynucleotide segment, but can be comprised of two or more individual segments interrupted by non-complementary sequences. These non-hybridizable sequences, if present, are linear. In addition, the complementary region of the nucleic acid probe can be flanked at the 3′- and 5′-termini by non-hybridizable sequences, such as those comprising the DNA or RNA of a vector into which the complementary sequence had been inserted for propagation. In either instance, the nucleic acid probe as presented as an analytical reagent will exhibit detectable hybridization at one or more points with target nucleic acids of interest. The nucleic acid probe sequence can be of any convenient or desired length, ranging from as few as a dozen to as many as 10,000 bases, and including oligonucleotides having less than about 50 bases (e.g., between 20 and 50 bases). The nucleic acid probe may be an oligonucleotide produced by solid-phase chemistry by a nucleic acid synthesizer. The RNA or DNA probe can be obtained in a variety of other conventional manners. It should be understood that in using the expressions RNA probe and DNA probe herein, it is not implied that all nucleotides comprised in the probe be ribonucleotides or 2′-deoxyribonucleotides. Therefore, one or more of the 2′-positions on the nucleotides comprised in the probe can be chemically modified, if desired, and provided there is no substantial interference with the hybridization assay. Likewise, in addition or alternatively to such limited 2′-deoxy modification, the nucleic acid probe can have in general any other modification along its ribose phosphate backbone provided there is no substantial interference with the hybridization assay.

In an embodiment, the nucleic acid probe may be labeled with an enzyme or other molecule that can be used to detect the nucleic acid molecule. In addition to the enzyme label, the nucleic acid probe may be labeled with either a detectable moiety or an immobilizable moiety. For example, the nucleic acid probe may be prepared by solid-phase chemistry using a nucleic acid synthesizer and may have a trityl-hexyl thiol derivatized 5′-end. The covalent attachment of the label to this moiety may be achieved by a number of well-known methods using a wide range of heterobifunctional reagents. For example, the method of Carlsson et al. (Biochem J. 173:723-737, 1978) may be used: the label is reacted with 3-[(2)-pyridyldithio]propionic acid N-hydroxysuccinimide ester (SPDP) to give a 2-pyridyl disulphide-activated label. This allows disulfide exchange with trityl-hexyl thiol derivatized described above to yield a labeled nucleic acid probe. Other approaches for labeling the nucleic acid probe will be apparent to one skilled in the art. Additionally, a wide range of labeled nucleic acids is available from commercial sources. Preferred labels include the enzymes alkaline phosphatase, peroxidase, β-galactosidase, nuclease P₁ and nuclease S₁; and the haptens digoxin, digoxygenin, fluorescein, fluorescein isothiocyanate, and biotin or biotin analogues.

Reverse Transcription Primers for Use in the Yeast/Mold Assay

Reverse Transcription primers for use in methods of amplifying Chitin Synthase class III genes or RNAs in the yeast/mold assay of the present invention include a non-T7 (NT7) primer having a length of between 10 and 30 nucleotides (e.g., 17 nucleotides) and having a sequence that is complementary to, and specific for, a sequence with at least 70% sequence identity (e.g., at least 80%, 85%, 90%, 95%, 97%, 99%, or 100% sequence identity) to the region corresponding to at least nucleotides 2865-2881 of one or more of SEQ ID NOs: 1-42 and 153. In a preferred embodiment, the NT7 primer has a sequence that is complementary to, and specific for, a sequence with at least 70% sequence identity (e.g., at least 80%, 85%, 90%, 95%, 97%, 99%, or 100% sequence identity) to the NT7 primer sequence shown in FIG. 9. More preferably the sequence is complementary to the NT7 primer shown in FIG. 9.

Reverse Transcription primers for use in methods of amplifying 1,3-β-glucan synthase (FKS1) genes or RNAs in the yeast/mold assay of the present invention include a primer having a length of between 10 and 27 nucleotides (e.g., 24 nucleotides) and having a sequence that is complementary to, and specific for, a sequence with at least 70% sequence identity (e.g., at least 80%, 85%, 90%, 95%, 97%, 99%, or 100% sequence identity) to the region corresponding to at least nucleotides 5361-5383 of one or more of SEQ ID NOs: 65-125 and 154.

Torch Oligomers for Use in the Yeast/Mold Assay

Torch oligomers for use in detecting amplified Chitin Synthase class III genes or RNAs in the yeast/mold assay of the present invention include oligos having lengths of between 10 and 27 nucleotides (e.g., 22 nucleotides) and having sequences that are complementary to, and specific for, sequences with at least 70% sequence identity (e.g., at least 80%, 85%, 90%, 95%, 97%, 99%, or 100% sequence identity) to the region corresponding to at least nucleotides 2907-2928 of one or more of SEQ ID NOs: 1-42 and 153. In a preferred embodiment, the Torch oligomer has a sequence that is complementary to, and specific for, a sequence with at least 70% sequence identity (e.g., at least 80%, 85%, 90%, 95%, 97%, 99%, or 100% sequence identity) to the torch oligomer sequence shown in FIG. 9. More preferably the sequence is complementary to the torch oligomer shown in FIG. 9.

Torch oligomers for use in detecting amplified 1,3-β-glucan synthase (FKS1) genes or RNAs in the yeast/mold assay of the present invention include an oligo having a length of between 10 and 30 nucleotides (e.g., 26 nucleotides) and having a sequence that is complementary to, and specific for, a sequence with at least 70% sequence identity (e.g., at least 80%, 85%, 90%, 95%, 97%, 99%, or 100% sequence identity) to the region corresponding to at least nucleotides 5409-5433 of one or more of SEQ ID NOs: 65-125 and 154.

T7 Provider Oligomers for Use in the Yeast/Mold Assay

T7 provider oligomers for use in amplifying Chitin Synthase class III genes or RNAs in the yeast/mold assay of the present invention include a T7 provider oligo having a length of between 10 and 25 nucleotides (e.g., 21 nucleotides) and having a sequence that is complementary to, and specific for, a sequence with at least 80% sequence identity to the region corresponding to at least nucleotides 2930-2950 of one or more of SEQ ID NOs: 1-42 and 153 in addition to a T7 promoter sequence. In a preferred embodiment, the T7 provider oligomer has a sequence that is complementary to, and specific for, a sequence with at least 70% sequence identity (e.g., at least 80%, 85%, 90%, 95%, 97%, 99%, or 100% sequence identity) to the T7 provider oligomer sequence shown in FIG. 9. More preferably the sequence is complementary to the T7 provider oligomer shown in FIG. 9.

T7 provider oligomers for use in amplifying 1,3-β-glucan synthase (FKS1) genes or RNAs in the yeast/mold assay of the present invention include an oligo having a length of between 10 and 32 nucleotides (e.g., 27 nucleotides) and having a sequence that is complementary to, and specific for, a sequence with at least 70% sequence identity (e.g., at least 80%, 85%, 90%, 95%, 97%, 99%, or 100% sequence identity) to the region corresponding to at least nucleotides 5435-5457 of one or more of SEQ ID NOs: 65-125 and 154.

Blocker Oligomers for Use in the Yeast/Mold Assay

Blocker oligomers for use in methods of amplifying Chitin Synthase class III genes or RNAs in the yeast/mold assay of the present invention include oligos having lengths of between 10 and 24 nucleotides (e.g., 18 nucleotides) and having sequences that are complementary to, and specific for, sequences with at least 70% sequence identity (e.g., at least 80%, 85%, 90%, 95%, 97%, 99%, or 100% sequence identity) to the region corresponding to nucleotides 2942-2959 of one or more of SEQ ID NOs: 1-42 and 153 and are optionally modified (e.g., a methoxy modification, such as at the 2′ position of ribose or at the 3′ end of the oligo; or a locked nucleic acid (LNA) modification, such as T or A having a methylene bridge connecting the 2′-O atom and the 4′-C atom of ribose) to either bind RNA or DNA specifically. In a preferred embodiment, the blocker oligomer has a sequence that is complementary to, and specific for, a sequence with at least 70% sequence identity (e.g., at least 80%, 85%, 90%, 95%, 97%, 99%, or 100% sequence identity) to the blocker oligomer sequence shown in FIG. 9. More preferably the sequence is complementary to the blocker oligomer shown in FIG. 9.

Blocker oligomers for use in methods of amplifying 1,3-β-glucan synthase (FKS1) genes or RNAs in the yeast/mold assay of the present invention include an oligo having a length of between 10 and 24 nucleotides (e.g., 20 nucleotides) and having a sequence that is complementary to, and specific for, a sequence with at least 70% sequence identity (e.g., at least 80%, 85%, 90%, 95%, 97%, 99%, or 100% sequence identity) to the region corresponding to at least nucleotides 5496-5514 of one or more of SEQ ID NOs: 65-125 and 154.

Displacer Oligomers for Use in the Yeast/Mold Assay

Displacer oligomers for use in methods of amplifying DNA (e.g., Chitin Synthase class III genes) in the yeast/mold assay of the present invention include oligos having lengths of between 10 and 24 nucleotides (e.g., 20 nucleotides) and having sequences that are complementary to, and specific for, sequences with at least 70% sequence identity (e.g., at least 80%, 85%, 90%, 95%, 97%, 99%, or 100% sequence identity) to the region corresponding to nucleotides upstream from nucleotide 2864 of one or more of SEQ ID NOs: 1-42 and 153 (e.g., sequences of between 10-24 nucleotides (e.g., 20 nucleotides) within the region of nucleotides 2750-2864 of SEQ ID NOs: 1-42 and 153). In a preferred embodiment, the displacer oligomer has a sequence that is complementary to, and specific for, a sequence with at least 70% sequence identity (e.g., at least 80%, 85%, 90%, 95%, 97%, 99%, or 100% sequence identity) to the displacer oligomer sequence shown in FIG. 9. More preferably the sequence is complementary to the displacer oligomer shown in FIG. 9.

Displacer oligomers for use in methods of amplifying 1,3-β-glucan synthase (FKS1) genes or RNAs in the yeast/mold assay of the present invention include an oligo having a length of between 10 and 24 nucleotides (e.g., 20 nucleotides) and having a sequence that is complementary to, and specific for, a sequence with at least 70% sequence identity (e.g., at least 80%, 85%, 90%, 95%, 97%, 99%, or 100% sequence identity) to the region corresponding to nucleotides upstream from nucleotide 5360 of one or more of SEQ ID NOs: 65-125 and 154 (e.g., sequences of between 10-24 nucleotides (e.g., 20 nucleotides) within the region of nucleotides 5250-5360 of SEQ ID NOs: 65-125 and 154).

### Application of the Yeast/Mold Assay to Clinical Samples

The yeast/mold assay of the invention can also be applied to clinical diagnostic purposes. In this embodiment, blood is collected from a patient and centrifuged in order to separate the blood into 3 layers (i.e., the red blood cells, the buffy coat, and the plasma). The plasma and the buffy coat layer are then collected and treated with a detergent. The resulting product is then treated with, for example, an enzyme, such as DNase I, in order to digest any non-fungal DNA which may be present. The chitin cell wall of the fungi is then digested, using Zymolase, for example, in order to release the fungal DNA for analysis. The DNA is then freed from bound protein by using, for example, Proteinase K digestion followed by detergent treatment. The DNA is then extracted by using phenol, for example, and then precipitated by using a chemical such as ethanol, for example. Subsequently, the DNA pellet is resuspended in distilled water. One is then ready to analyze the sample using the primers.

The extracted DNA can be analyzed by one or more amplification methods known in the art and described herein, including, for example, replicase-mediated amplification, PCR, LCR, SDA, and RTMA, and one or more of the mold and/or yeast/mold-specific primers and oligos described herein (see, e.g., FIGS. 2 and 5, respectively).

After the DNA-fungal-specific product is made, it may then be analyzed using either a liquid assay or a solid support-based assay (e.g., an agarose gel). A negative control (e.g., no DNA is added to one set of control tubes, so no DNA product should be made) and/or a positive control can be run, if desired.

### Application of the Yeast/Mold Assay to Industrial Samples

The methods disclosed herein can also be used to detect yeast or mold present in industrial processes, such as pharmaceutical manufacturing, transportation fuel manufacturing (e.g., fuel ethanol manufacturing and hydrocarbon fuels), fermentation systems, and industrial processes requiring water-cooling towers, as well as in industrial reagents, fluids (e.g., metal working fluids and water-based coolants), lubricants, holding tanks, or other liquid or solid components, and products, e.g., petroleum products. Fungal contaminants present in industrial processes and reagents can cause obstruction of fluid delivery lines or chemical deterioration in machinery that result in dangerous situations, as well as poor performance and costly repairs. In addition, fungal growth in industrial systems can be responsible for corrosion, slime formation, foul odors, and decreases in fluid function.

The primers and oligos described herein can be used to detect target polynucleotides that are associated with yeast and/or mold present in a sample from an industrial process or product. The methods of the invention may be used to detect genes or RNAs, such as chitin synthase (e.g., chitin synthase class III) or 1,3-β-glucan synthase (FKS1) genes or RNAs, associated with the yeast and/or mold disclosed herein.

### Application of the Yeast/Mold Assay to Water-Based Samples

The methods disclosed herein can also be used to provide a rapid and sensitive diagnostic test for the presence and enumeration of yeast and/or mold in any water sample, including, but not limited to, swimming pools, aquatic parks, wells, home drinking water, reservoirs, beaches, lakes, oceans, fish and shellfish farms, agricultural water, dialysis water, medication reconstitution water, water treatment facilities, cruise ships, and bottled water.

The primers and oligos described herein can be used to detect target polynucleotides that are associated with yeast and/or mold present in a water-based sample. The methods of the invention may be used to detect genes or RNAs, such as chitin synthase (e.g., chitin synthase class III) or 1,3-β-glucan synthase (FKS1) genes or RNAs, associated with the yeasts and/or molds disclosed herein.

### Application of the Yeast/Mold Assay to Food and Environmental Samples

Yeast and/or mold present in food and/or environmental samples may be detected by the methods of the present invention. Food samples include liquid and solid food, such as beef, pork, sheep, bison, deer, elk, poultry, fish, produce, dairy products, dry goods, ready-made food products, raw and processed foods, and feed products, and ingredients, such as dairy items, vegetables, meat and meat by-products, and waste. Environmental samples include but are not limited to, e.g., environmental material, such as surface matter, soil, sludge, samples taken by impingers and filtration, processing instruments, apparatus, equipment, utensils, disposable and non-disposable items, and materials present in and on buildings and in hospital settings, including, e.g., building and other materials, such as the paper on gypsum wallboard (drywall), ceiling tiles, wood products, paint, wallpaper, carpeting, ventilation systems (e.g., system components, such as filters, insulation, ductwork, and coils/fins), some furnishings, books/papers, clothes and other fabrics, concrete, fiberglass insulation, ceramic tiles, bathroom tile grout, shower stalls, toilets, bathtubs, cabinets, medical equipment and systems.

The primers and oligos described herein can be used to detect one or more target polynucleotides associated with yeast and/or mold (e.g., chitin synthase (e.g., chitin synthase class III) or 1,3-β-glucan synthase (FKS1) genes or RNAs) present in food and environmental samples using the methods of the present invention.

### Kits of the Invention

The invention also features a kit for carrying out the described methods according to the present invention described herein. The kit includes nucleic acid probes or primers that may be labeled, reagents and containers for carrying out the hybridization assay, positive and negative control reagents, and instructions for performing the assay.

Some kits contain at least one target capture oligomer for hybridizing to a target nucleic acid. Some kits for detecting the presence or abundance of two or more target nucleic acids contain two or more target capture oligomers each configured to selectively hybridize to each of their respective target nucleic acids.

Some kits contain at least one first amplification oligomer (e.g., a SMART tag NT7 primer) for hybridizing to a target nucleic acid. Some kits for detecting the presence or abundance of two or more target nucleic acids contain two or more first amplification oligomers, each configured to selectively hybridize to their respective target nucleic acids.

Some kits contain chemical compounds used in performing the methods herein, such as enzymes, substrates, acids or bases to adjust pH of a mixture, salts, buffers, chelating agents, denaturants, sample preparation agents, sample storage or transport medium, cellular lysing agents, total RNA isolation components and reagents, partial generalized RNA isolation components and reagents, solid supports, and other inorganic or organic compounds. Kits may include any combination of the herein mentioned components and other components not mentioned herein. Components of the kits can be packaged in combination with each other, either as a mixture or in individual containers. It will be clear to skilled artisans that the invention includes many different kit configurations.

The kits of the invention may further include additional optional components useful for practicing the methods disclosed herein. Exemplary additional components include chemical-resistant disposal bags, tubes, diluent, gloves, scissors, marking pens, and eye protection.

### EXAMPLE

The present invention can be illustrated by the use of the following non-limiting example:

### Example 1

### Reverse Transcription-Mediated Amplification (RTMA)/PCR for Yeast and Mold Assay, Single Copy Gene Target

The yeast/mold assay of the invention may be performed using nucleic acid amplification methods known in the art (e.g., those methods described in WO 2007/146154 and US 2001-00323362, which are incorporated by reference herein in their entirety) using the nucleic acid probes and primers described herein. An RTMA assay for discriminating between yeast and/or mold in a sample is shown in FIG. 8 and described in detail below.

Since the yeast/mold assay attempts to detect a considerable amount of yeast and mold species, optional features of the assay can be used to reduce background and prevent detection of environmental contaminants once the sample is run in an open environment. The background reduction technology (BRT) used to control environmental contamination and decrease false-positive rates may include the following, optional, modifications:

a) SMART oligos: These oligos are designed in a way that the 5′ end of the oligos carries a tag sequence that is complementary to the 3′ end of the oligo and closes the oligo at the amplification temperature of 42° C. Therefore a SMART tag oligo prevents access to the target specific part of the oligo at lower temperatures and reduces false-positive rates of contaminations that come into the assay after target capture. The assay described below may include the use of SMART tag oligos.

b) non-target related tag: a non-target related sequence is inserted into the non-T7 (NT7) primer, between target specific sequence and SMART tag. By introducing a tag sequence into the NT7 primer, the system can be amplified with a non-target related primer after a first reverse transcription reaction using the tagged NT7 primer. This greatly reduces the effect of contaminating nucleic acids introduced into the system during or after target capture and increases amplification efficiency by improving the tag sequence.

Taken together, the SMART tag NT7 primer may contain both a SMART tag as well as a non-target related tag sequence. The SMART displacer only has a SMART tag since no displacement is needed after the initial reverse transcription reaction to generate a cDNA.

1) Target Capture and Primer Annealing:

The fungal organism needs to be lysed to access the genomic DNA and RNA target(s). Lysis could be mechanical (e.g., beads) or chemical (e.g., alkaline lysis). Another important step to allow efficient capture of the genomic DNA and RNA target(s) is the denaturation of the DNA and RNA, thereby separating complementary DNA strands. Efficient denaturation can be either achieved by heat treatment (>85° C.) or treatment with high alkaline (needs to be neutralized with acid before target capture).

The next step in an RTMA approach is pre-annealing of the NT7 primer, the displacer and a blocker. The blocker is a 3′ blocked methoxy oligo that is annealed together with the NT7 primer and the displacer. An optional target capture (TC) oligo may also be annealed to the denatured single, stranded DNA or RNA target. For efficient capture of single stranded DNA a wobble capture oligo may be used as the TC oligo. The target capture (TC) oligo consists of a random GU mixture that is 18 nucleotides long, has a spacer region of 3 T and 30 A that allows binding of the polT tails of magnetic beads to allow the separation and capture of nucleic acids from the sample. To further reduce false-positive rate the wobble target capture oligo can be closed by a 15T long SMART tag that allows binding to the 5′ polyA tail. Annealing of all these oligos to the target is most efficiently done in lysis buffer at 60° C. for 10 (to up to, e.g., 60 minutes). This allows the SMART tags, if present, to open and the target specific regions to bind to target if present. After the annealing the sample is cooled to RT and should not be heated higher than 42° C. from this point on. Cooling to RT (a temperature of about 5° C. to about 30° C., in particular from about 10° C. to about 27° C. (e.g., about 23-27° C.)) forces the SMART tag oligos, if present, that are not bound to target to close and therefore renders them incapable of priming at 42° C. All steps up to this point should be done in a sterile contained environment, yet after the SMART tags are closed, target capture and amplification can be run in an open system.

For an amplification approach (e.g., a PCR approach), only the target capture would be annealed to the target in this step.

If a TC oligo is used in the assay, magnetic beads with a poly T tail may be added to the reaction and used to separate nucleic acids and annealed primers from the rest of the sample. Different platforms allow a semi-automation of this magnetic separation (e.g., KingFisher or DTS). After magnetic separation the beads containing the captured nucleic acids may be introduced into the amplification reaction.

2) Reverse Transcription-Mediated Amplification (RTMA)

Target nucleic acids generated in the RT step are now amplified at 42° C., with the non-target related tag primer and a target specific T7 provider. The T7 provider is 3′ blocked and is not a primer. Specific RNA amplicon can be detected in a real-time format using a torch that is designed to detect universally conserved regions for all yeast and mold. Yeast and molds can be distinguished in a real-time assay using different fluorophores to mark the different torches.

In a PCR assay, the captured target would now be amplified using redundant forward and reverse primers and a molecular probe (beacon or TaqMan probe) to monitor amplification in real-time.

To provide a sterile environment for the first part of the assay, lysis buffer as well as oligos can be base-treated and then neutralized. This treatment protocol can be further extended to ensure the destruction of contaminating DNA or RNA.

Other details of the RTMA procedure can be found in, e.g., U.S. Patent Application Publication No. 2011-0014623, which is incorporated herein by reference.

### Example 2

### Detection of Gene Targets Using RTMA for a Mold and Yeast Assay

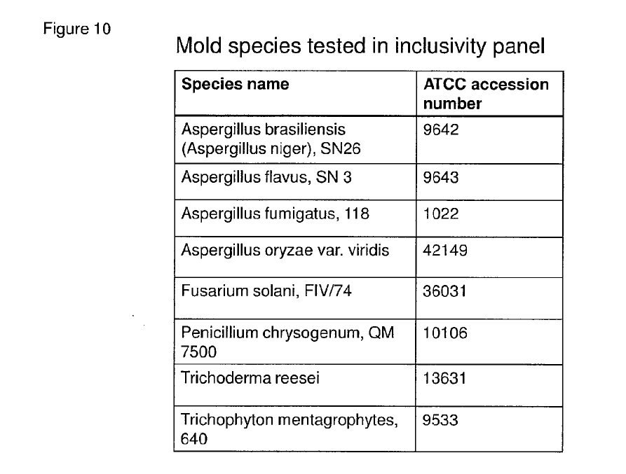

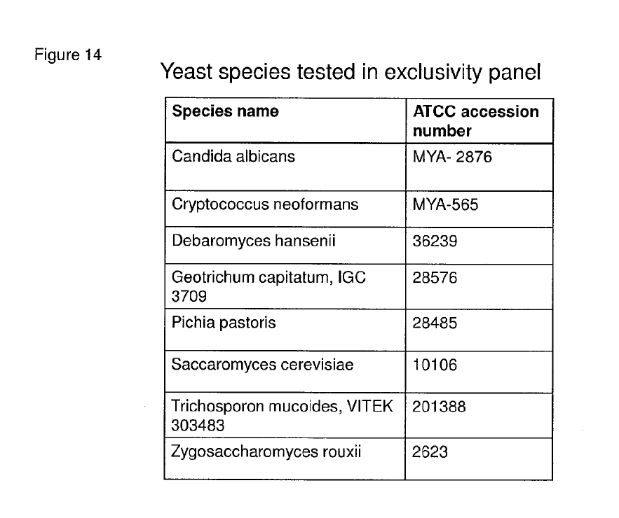

Using the RTMA method described in Example 1 (without the optional SMART tags), DNA targets in various mold and yeast species were detected in an inclusivity panel for molds and an exclusivity panel for yeasts. The following oligo sequences were used in this assay: T7 provider (7.5 pmol/reaction), torch (8 pmol/reaction), NT7 primer (5 pmol/reaction), displacer (0.5 pmol/reaction), and blocker (0.5 pmol/reaction), where these sequences are provided in FIG. 9. For amplification, APTIMA® buffer and enzyme (from Gen-Probe Inc., San Diego, Calif.) were added in a reaction volume of 40 μl. The assay included DNA from various mold and yeast species, as provided in FIGS. 10 and 14. Amount of amplified target DNA was calculated based on genome length and ng of DNA isolated from fungi.

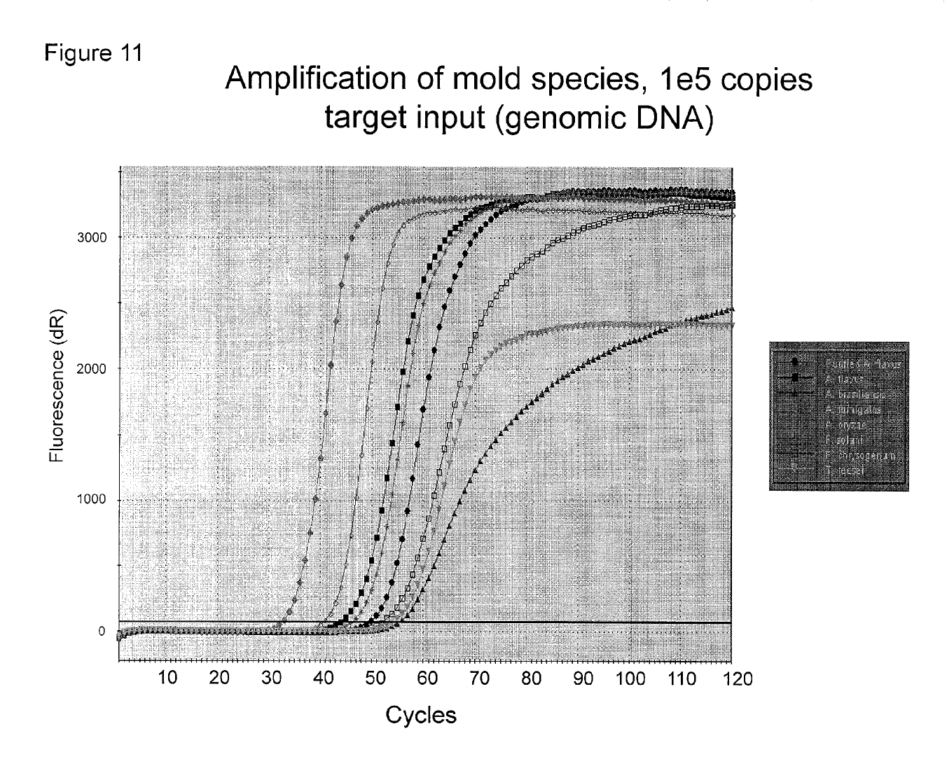

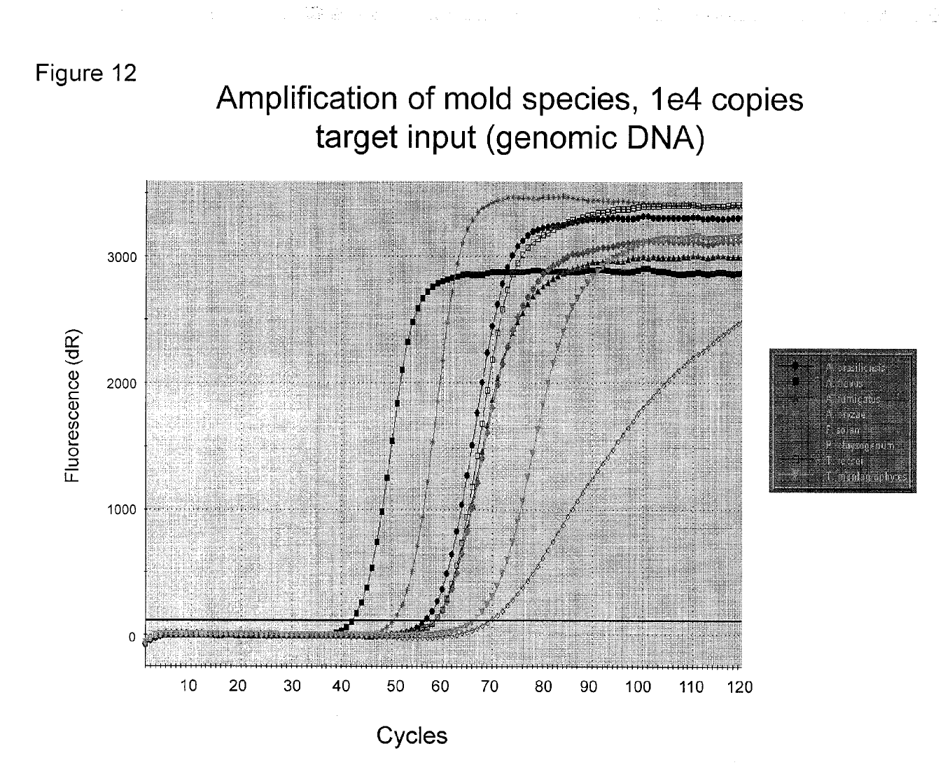

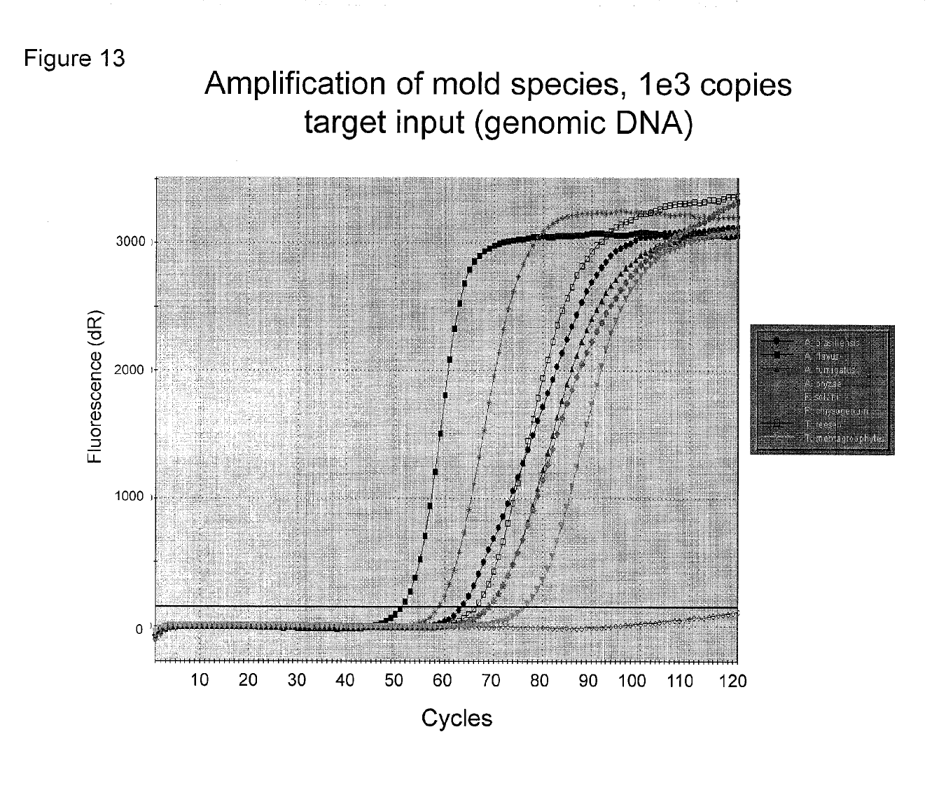

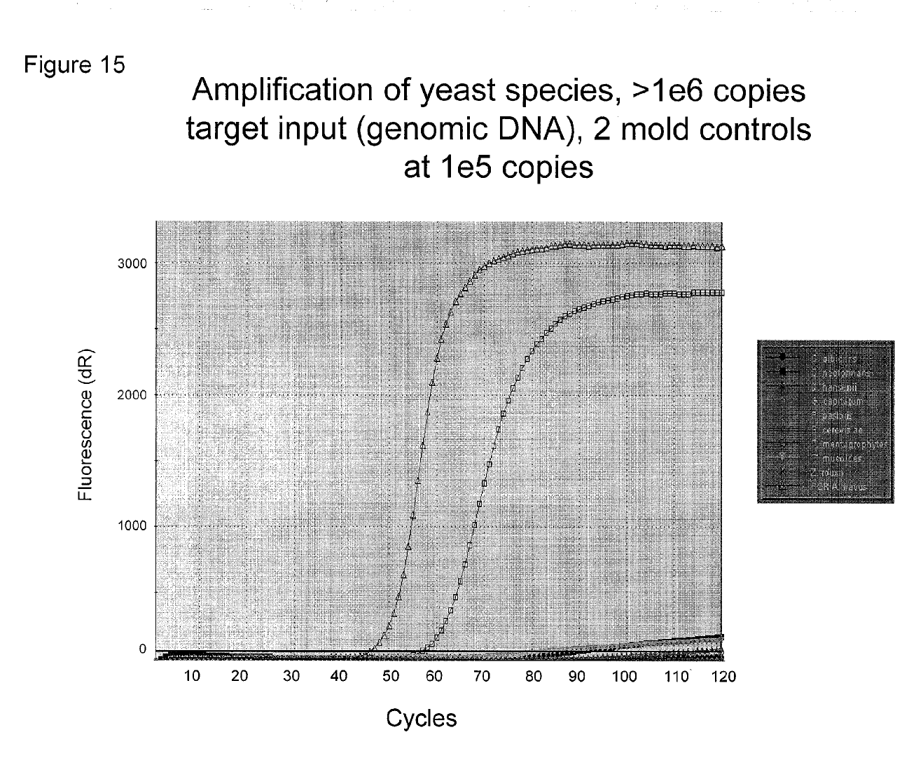

Using the Chitin Synthase class III oligo sequences of FIG. 9, the assay provides amplified products for the mold species in the inclusivity panel (FIG. 10) but not the yeast species in the exclusivity panel (FIG. 14). Various species of mold were amplified using 1×10⁵, 1×10⁴, or 1×10³ copies of genomic DNA as input (FIGS. 11-13, respectively). In contrast, yeast species (using 1×10⁶ copies of genomic DNA as the target input) were not amplified in this assay, where only positive control using two mold species were amplified (FIG. 15). Taken together, the methods, compounds, and kits disclosed herein can be used to detect and distinguish between yeast or mold in a sample.

### OTHER EMBODIMENTS

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

1. A method for detecting and distinguishing between yeast or mold in a sample suspected of being contaminated with said yeast or mold comprising:
a) contacting nucleic acid molecules from said sample with a mold-specific probe or a yeast or mold-specific probe under hybridization conditions, wherein
i) said mold-specific probe is capable of specifically binding to a mold-specific region comprising at least 17 contiguous base pairs within an endogenous chitin synthase gene of said mold but not to a yeast or mold-specific region of the endogenous chitin synthase gene, wherein said mold-specific region shares greater than 72% sequence identity between two or more different species of said mold; and
ii) said yeast or mold-specific probe is capable of specifically binding to a yeast or mold-specific region comprising at least 17 contiguous base pairs within an endogenous 1,3-β-glucan synthase gene of said yeast or mold, wherein said yeast or mold-specific region shares greater than 70% sequence identity between two or more different species of said yeast or mold; and
b) detecting hybridization between said mold-specific or said yeast or mold-specific probe and said nucleic acid molecules, wherein
i) hybridization of said mold-specific probe with said nucleic acid molecule indicates the presence of said mold in said sample; or
ii) hybridization of said yeast or mold-specific probe with said nucleic acid molecule indicates the presence of said yeast or mold in said sample.

2. The method of claim 1, wherein said endogenous chitin synthase gene specifically bound by said mold-specific probe is a chitin synthase class III gene.

3. The method of claim 1, wherein said endogenous 1,3-β-glucan synthase gene specifically bound by said yeast or mold-specific probe is a 1,3-β-glucan synthase gene.

4. The method of claim 1, said mold-specific region comprises at least about 17 contiguous base pairs within the region of nucleotides 2859 to 2968 of the chitin synthase class III gene, or a ribonucleic acid (RNA) product thereof or said yeast or mold-specific region comprises at least about 17 contiguous base pairs within the region of nucleotides 5360 to 5515 of the 1,3-β-glucan synthase 1 gene, or a ribonucleic acid (RNA) product thereof.

5. (canceled)

6. The method of claim 1, comprising incubating the mold-specific and yeast or mold-specific probes with the nucleic acid molecules under hybridization conditions, wherein
i) hybridization of said mold-specific probe and said yeast or mold-specific probe with said nucleic acid molecules indicates the presence of said mold only or said yeast and said mold in said sample; and
ii) hybridization of said mold-specific probe, but not said yeast or mold-specific probe, with said nucleic acid molecule indicates the presence of said mold but not said yeast in said sample; and
iii) hybridization of said yeast or mold-specific probe, with said nucleic acid molecule indicates the presence of said yeast or mold.

7. The method of claim 6, wherein a difference in hybridization signal intensity between said mold-specific probe relative to said yeast or mold-specific probe indicates the presence of said yeast in said sample.

8. The method of claim 1, wherein, prior to said contacting, said method comprises amplifying said nucleic acid molecules from said sample.

9. The method of claim 8, wherein said amplifying comprises:
(a) contacting said sample comprising said nucleic acid molecules with a first amplification primer having a target sequence specific for a 5′ region of said endogenous chitin synthase gene or RNA of said mold and/or a second amplification primer having a target sequence specific for a 5′ region of said 1,3-β-glucan synthase gene or RNA; and
(b) performing a nucleic acid amplification reaction to produce a first and/or second amplification product, respectively.

10. The method of claim 9, wherein said first and/or second amplification product comprises a region of at least about 15 contiguous base pairs that are conserved between different fungal microorganisms.

11. The method of claim 10, wherein said region is conserved between different yeast or different molds.

12. (canceled)

13. The method of claim 10, wherein said region comprises about 20 to about 100 base pairs.

14. The method of claim 13, wherein said region comprises about 60 base pairs.

15. The method of claim 1, wherein said mold-specific probe comprises a sequence having at least 70% sequence identity to a complementary sequence comprising at least 17 contiguous nucleotides within the sequence of SEQ ID NO:153 or said yeast or mold-specific probe comprises a sequence having at least 70% sequence identity to a complementary sequence comprising at least 15 contiguous nucleotides within the sequence of SEQ ID NO:154.

16-17. (canceled)

18. The method of claim 1, wherein said mold-specific probe comprises a sequence having at least 70% sequence identity to a complementary sequence comprising 17 to 100 contiguous nucleotides within nucleotides 2850 to 2970 of the sequence of any one of SEQ ID NOs:1-42 or said yeast or mold-specific probe comprises a sequence having at least 70% sequence identity to a complementary sequence comprising 17 to 100 contiguous nucleotides within nucleotides 5360 to 5515 of the sequence of any one of SEQ ID NOs:65-125.

19-22. (canceled)

23. The method of claim 1, wherein said yeast or mold-specific probe binds to said yeast or mold-specific region within said yeast and mold.

24. The method of claim 1, wherein said method distinguishes between the presence of said mold and said yeast in said sample.

25. The method of claim 1, wherein said mold is selected from phylum Ascomycota, phylum Basidomycota, phylum Chytridiomycota, phylum Zygomycota, and phylum Oomycota in the Stramenopila kingdom.

26. The method of claim 25, wherein:
a) said mold of phylum Ascomycota is Ajellomyces spp., Alternaria spp., Aschersonia spp., Aspergillus spp., Arthroderma spp., Ascochyta spp., Bipolaris spp., Blastomyces spp., Botryotinia spp., Chaetomium spp., Cladosporium spp., Coccidioides spp., Curvularia spp., Emericella spp., Emmonsia spp., Epicoccum spp., Exophiala spp., Fusarium spp., Geomyces spp., Geotrichum spp., Gibberella spp., Histoplasma spp., Magnaporthe spp., Metarhizium spp., Monascus spp., Mycospaerella spp., Nectria spp., Neosartorya spp., Neurospora spp., Paecilomyces spp., Paracoccidioides spp., Penicillium spp., Phaeosphaeria spp., Phialemonium spp., Podospora spp., Pyrenophora spp., Sclerotinia spp., Scopulariopsis spp., Sporothrix spp., Stachybotrys spp., Stemphylium spp., Talaromyces spp., Trichophyton spp., Trichothecium spp., Tricoderma spp., Tuber spp., Uncinocarpus spp., or Verticillium spp.;
b) said mold of phylum Basidomycota is Moniliophthora spp., Sporobolomyces spp., Trichosporon spp., or Ustilago spp.; or
c) said mold of phylum Zygomycota is Absidia spp., Amylomyces spp., Pilaira spp., Rhizomucor spp., Rhizopus spp., or Zygomycetes spp.

27-28. (canceled)

29. The method of claim 1, wherein said yeast is selected from phylum Ascomycoata and phylum Basidomycota.

30. The method of claim 29, wherein:
a) said yeast of phylum Ascomycota is Candida spp., Clavispora spp., Debaryomyces spp., Dekkera spp., Kluyveromyces spp., Pichia spp., Saccaromyces spp., Torulaspora spp., Vanderwaltozyma spp., Yarrowia spp., or Zygosaccharomyces spp.; or
b) said yeast of phylum Basidomycota is Cryptococcus spp., or Rhodotorula spp.

31. (canceled)

32. The method of claim 1, wherein said sample is selected from an agricultural product or process, an industrial product or process, an environmental product or process, a food product or process, a pharmaceutical product or process, a water-based product or process, a wood product, a textile, and a tissue product.

33. The method of claim 32, wherein:
a) said agricultural product is selected from a fruit, a vegetable, a grain, forage, a silage, a juice, a wood, a flower, and a seed;
b) said fruit is selected from a tomato, a pepper, a grape, an apple, an orange, a lemon, and a berry; or
c) said tissue product is blood, urine, or serum.

34-35. (canceled)

36. The method of claim 1, wherein said method comprises lysing cells present in said sample.

37. The method of claim 36, wherein said lysing comprises mechanical or chemical lysis.

38. The method of claim 1, wherein prior to step a), said method comprises denaturing complementary strands of said nucleic acid molecules.

39. The method of claim 1, wherein said nucleic acid molecules comprise deoxyribonucleic acid molecules (DNA).

40. A kit comprising (i) a first container comprising the mold-specific probe of claim 1, (ii) a second container comprising the yeast or mold-specific probe of claim 1, and (iii) instructions for performing the method of claim 1 for detecting and discriminating between mold or yeast in a sample.

41. The kit of claim 40, wherein said mold-specific probe and said yeast or mold-specific probe comprise different detectable labels.

42. A kit comprising (i) a container comprising the mold-specific probe of claim 1 and the yeast or mold-specific probe of claim 1, wherein the mold-specific and yeast or mold-specific probes comprise different detectable labels; and (ii) instructions for performing the method of claim 1 for detecting and discriminating between mold or yeast in a sample.
